SEQUENCE LISTING (29 sequences)

SEQ ID NO: 1 - protein, 162 aa, Homo sapiens
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 2 - protein, 135 aa, Homo sapiens
MVLGTIDLCSCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 3 - protein, 114 aa, Homo sapiens
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 4 - DNA, 489 bp, Homo sapiens
atgagaatttcgaaaccacatttgagaagtatttccatccagtgctacttgtgtttacttctaaacagtcattttctaactgaagctggcattcatgtcttcattttgggctgtttcagtgcagggcttcctaaaacagaagccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttcttga

SEQ ID NO: 5 - DNA, 408 bp, Homo sapiens
atggtattgggaaccatagatttgtgcagctgtttcagtgcagggcttcctaaaacagaagccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttcttga

SEQ ID NO: 6 - DNA, 345 bp, Homo sapiens
aactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttcttga

SEQ ID NO: 7 - protein, 9 aa, Artificial sequence
  Synthetic polypeptide
NVISDLKKI

SEQ ID NO: 8 - protein, 6 aa, Artificial sequence
  Synthetic polypeptide
DTVENL

SEQ ID NO: 9 - protein, 8 aa, Artificial sequence
  Synthetic polypeptide
HIVQMFIN

SEQ ID NO: 10 - protein, 11 aa, Artificial sequence
  Synthetic polypeptide
YGRKKRRQRRR

SEQ ID NO: 11 - protein, 12 aa, Artificial sequence
  Synthetic polypeptide
RRQRRTSKLMKR

SEQ ID NO: 12 - protein, 27 aa, Artificial sequence
  Synthetic polypeptide
GWTLNSAGYLLGKINLKALAALAKKIL

SEQ ID NO: 13 - protein, 33 aa, Artificial sequence
  Synthetic polypeptide
KALAWEAKLAKALAKALAKHLAKALAKALKCEA

SEQ ID NO: 14 - protein, 16 aa, Artificial sequence
  Synthetic polypeptide
RQIKIWFQNRRMKWKK

SEQ ID NO: 15 - protein, 9 aa, Artificial sequence
  Synthetic polypeptide
RKKRRQRRR

SEQ ID NO: 16 - protein, 8 aa, Artificial sequence
  Synthetic polypeptide
RKKRRQRR

SEQ ID NO: 17 - protein, 11 aa, Artificial sequence
  Synthetic polypeptide
YARAAARQARA

SEQ ID NO: 18 - protein, 11 aa, Artificial sequence
  Synthetic polypeptide
THRLPRRRRRR

SEQ ID NO: 19 - protein, 11 aa, Artificial sequence
  Synthetic polypeptide
GGRRARRRRRR

SEQ ID NO: 20 - protein, 5 aa, Artificial sequence
  Synthetic polypeptide
  MISC_FEATURE (5)..(5): This residue may be repeated.
GSGGS

SEQ ID NO: 21 - protein, 4 aa, Artificial sequence
  Synthetic polypeptide
  MISC_FEATURE (4)..(4): This residue may be repeated.
GGGS

SEQ ID NO: 22 - protein, 5 aa, Artificial sequence
  Synthetic polypeptide
  MISC_FEATURE (1)..(1): This residue may be repeated.
  MISC_FEATURE (2)..(2): This residue may be repeated.
  MISC_FEATURE (3)..(3): This residue may be repeated.
  MISC_FEATURE (4)..(4): This residue may be repeated.
  MISC_FEATURE (5)..(5): This residue may be repeated.
GSGSG

SEQ ID NO: 23 - protein, 5 aa, Artificial sequence
  Synthetic polypeptide
  MISC_FEATURE (1)..(5): This stretch of residues may be repeated.
  MISC_FEATURE (5)..(5): This residue may be repeated.
GSGGS

SEQ ID NO: 24 - protein, 5 aa, Artificial sequence
  Synthetic polypeptide
  MISC_FEATURE (1)..(5): This stretch of residues may be repeated.
  MISC_FEATURE (4)..(4): This residue may be repeated.
GSGSG

SEQ ID NO: 25 - protein, 4 aa, Artificial sequence
  Synthetic polypeptide
  MISC_FEATURE (1)..(4): This stretch of residues may be repeated.
  MISC_FEATURE (4)..(4): This residue may be repeated.
GGGS

SEQ ID NO: 26 - protein, 4 aa, Artificial sequence
  Synthetic polypeptide
GGSG

SEQ ID NO: 27 - protein, 5 aa, Artificial sequence
  Synthetic polypeptide
GGSGG

SEQ ID NO: 28 - protein, 5 aa, Artificial sequence
  Synthetic polypeptide
GSGGG

SEQ ID NO: 29 - protein, 5 aa, Artificial sequence
  Synthetic polypeptide
GSSSG

(19) United States
(12) Patent Application Publication
(10) Pub. No.: US 2018/0360977 A1
(43) Pub. Date: Dec. 20, 2018

(54) Interleukin-15 Compositions and Uses Thereof

(71) Applicant: ARMO BioSciences, Inc., Redwood City, CA (US)

(72) Inventors: Scott Alan McCauley, Brisbane, CA (US); John Brian Mumm, Los Altos Hills, CA (US); Ivan Ho Chan, Redwood City, CA (US)

(21) Appl. No.: 16/060,389

(22) PCT Filed: Dec. 15, 2016
(86) PCT No.: PCT/US16/67042

§ 371 Date: Jun. 7, 2018

Related U.S. Application Data
(60) Provisional application No. 62/270,447, filed on Dec. 21, 2015.

(51) Int. Cl. A61K 47/60; A61K 38/20; A61K 9/00

(52) U.S. Cl. CPC A61K 47/60; A61K 9/0019; A61K 38/2086

(57) ABSTRACT

Pegylated interleukin-15—related molecules and the identification thereof are described. The pegylated interleukin-15 molecules exhibit properties and characteristics that make them candidates for therapeutic use. Pharmaceutical compositions and methods of use are also described herein.

1C. Mature human IL-15 Protein
NWVNVISDLKKIEDLIQSMHIDATLYTESDVHPSCKVTAMKCFLLELQVI
SLESGDASIHDTVENLIILANNSLSSNGNVTESGCKECEELEEKNIKEFL
QSFVHIVQMFINTS

### CROSS-REFERENCE TO RELATED APPLICATION

This application claims priority benefit of U.S. Provisional Application Serial No. 61/270,447, filed Dec. 21, 2015, which application is incoroproated by reference herein in its entirety.

### FIELD OF THE INVENTION

The present invention relates to, among other things, pegylated interleukin-15 and uses thereof.

### INTRODUCTION

Interleukin-15 (IL-15) is a cytokine involved in the stimulation of cytolytic activity, cytokine secretion, proliferation and survival of NK cells, CD8+memory T-cells and naïve CD8+cells (see Fehniger, et al., J Immunol 162:4511-20 (1999)). As a pleiotropic cytokine, it plays important roles in innate and adaptive immunity (see Lodolce, et al., Cytokine Growth Factor Rev 13(6):429-39 (December 2002)) and Alves, et al., Blood 102:2541-46 (2003)).

IL-15 is constitutively expressed by a large number of cell types, including macrophages, monocytes, dendritic cells and fibroblasts (Grabstein, et al., Science 264(5161):965-68 (May 1994)). Expression of IL-15 can be stimulated by, for example, cytokines (e.g., GM-CSF), double-stranded mRNA, unmethylated CpG oligonucleotides, lipopolysaccharide through Toll-like receptors, and interferons (e.g., IFN-γ), or after infection of, for example, monocytes with herpes virus, Mycobacterium tuberculosis and Candida albicans (Bamford, et al., J Immunol 160(9):4418-26 (May 1998)).

IL-15 binds to a specific receptor complex on T-cells and NK cells. IL-15 and IL-15Rα are co-expressed on activated dendritic cells and on monocytes, and IL-15 functions in a complex with IL-15Rα (Bergamaschi, et al., J Biol Chem 283:4189-99 (2008)). IL-15/IL-15α bind as a heterodimer to two chains on T-cells and NK cells—IL-2R13 (also referred to as IL-15Rβ; CD122) and γc (also referred to as IL-2RG; CD132; γ-c; common γ-chain) molecules. The I and γc chains are shared between IL-2 and IL-15 and are essential for the signaling of these cytokines (Giri et al., EMBO J. 13:2822-30 (1994) and Giri et al., EMBO J. 14:3654-3663 (1995)).

Consistent with the sharing of the IL-2/IL-15βγc receptor complex, IL-15 has been shown to mediate many functions similar to those of IL-2 in vitro. They share many biological activities and exhibit similar contributions to the survival of T lymphocytes (see Waldmann, et al., Annu Rev Immunol 17:19-49 (1999)). It is believed that the biological differences between IL-2 and IL-15 are likely due to, for example, their different production sites, their strength of association with membrane receptor proteins, termed IL-2α and IL-15Rα, respectively, and the regulation of these extra receptor molecules. IL-2 and IL-15 play a role in regulating the number of CD8+memory cells.

Despite the fact that IL-15 has been implicated in a number of diseases, disorders and conditions, including, for example, certain viral disorders and cancerous conditions, no IL-15—related agent is currently commercially available. Thus, a safe and effective IL-15 agent would address a heretofore unmet medical need.

### SUMMARY

The present disclosure relates to pegylated IL-15 compositions and uses thereof. The terms “IL-15”, “IL-15 polypeptide(s),” “IL-15-agent(s)”, “IL-15 molecule(s)” and the like are intended to be construed broadly and include, for example, human and non-human IL-15—related polypeptides, including homologs, variants (including muteins), and fragments thereof, as well as IL-15 polypeptides having, for example, a leader sequence (e.g., a signal peptide). More particularly, the present disclosure is drawn to certain pegylated IL-15 agents having at least one property or other characteristic (e.g., extended half-life) that makes them superior to other IL-15 molecules and thus more beneficial from a therapeutic perspective.

Mature human IL-15 is a 114 amino acid monomeric polypeptide. Two transcripts have been reported, one with a 48 amino acid signal peptide (Long Signal Peptide; LSP) (FIG. 1A; SEQ ID NO:1), and the other with a 21 amino acid signal peptide (Short Signal Peptide; SSP) (FIG. 1B; SEQ ID NO:2), both of which produce the same mature protein (FIG. 1C; SEQ ID NO:3). The present disclosure contemplates embodiments wherein the mature hIL-15 protein is pegylated with one or more of the PEG moieties described herein. In certain embodiments, a PEG moiety is attached at the N-terminus of hIL-15, while in other embodiments it is attached at the C-terminus, and in still further embodiments it is attached at one or more residues other than the N-terminus and the C-terminus (i.e., at one or more of residues 2-113 of hIL-15).

Certain embodiments of the present disclosure comprise IL-15 muteins, which may be produced recombinantly, pegylated with one or more of the PEG moieties described herein. As set forth herein, mature human IL-15 is described as comprising four helices (A-D), also referred to as inter-helices junctions, linked by three distinct amino acid segments (AB Loop; B/C Turn; and C/D Loop). Amino acid residues and regions of the IL-15 helices and inter-helices junctions that can be mutated and/or modified to facilitate the attachment of the PEG moieties are described in detail hereafter. In certain embodiments, a PEG moiety is attached at the N-terminus of an IL-15 mutein, while in other embodiments it is attached at the C-terminus of an IL-15 mutein, and in still further embodiments it is attached at one or more residues other than the N-terminus and the C-terminus of an IL-15 mutein.

Chemistries currently exist for pegylation of, for example, a polypeptide's N-terminus, lysine residues, cysteine residues, histidine residues, arginine residues, aspartic acid residues, glutamic acid residues, serine residues, threonine residues, tyrosine residues, and C-terminus.

In particular embodiments, the present disclosure contemplates pegylated IL-15 peptides comprising the amino acid sequence of FIG. 1C (SEQ ID NO:3), wherein the peptides comprise at least one amino acid substitution, deletion or addition, and wherein the substitution(s), deletion(s) or addition(s) does not, for example, adversely affect solubility or immunogenicity. The present disclosure also contemplates peptides having at least 80%, at least 85%, at least 90%, at least 91%, at least 92%, at least 93%, at least 94%, at least 95%, at least 96%, at least 97%, at least 98%, or at least 99% sequence identity to the amino acid sequence of FIG. 1C (SEQ ID NO:3). In addition, in some embodiments, such pegylated IL-15 molecules have at least 60, at least 70, at least 80, at least 90, at least 95, at least 100, at least 101, at least 102, at least 103, at least 104, at least 105, at least 106, at least 107, at least 108, at least 109, at least 110, at least 111, at least 112, or at least 113 amino acid residues.

In particular embodiments, the present disclosure contemplates pegylated IL-15 peptides having a bioactivity greater than the bioactivity of FIG. 1C (SEQ ID NO:3). In other particular embodiments, the present disclosure contemplates pegylated IL-15 peptides having a bioactivity comparable to the bioactivity of FIG. 1C (SEQ ID NO:3). In still further particular embodiments, the present disclosure contemplates pegylated IL-15 peptides having a bioactivity less than the bioactivity of FIG. 1C (SEQ ID NO:3). Bioactivity is just one of several parameters and characteristics that may be used to assess the usefulness of the pegylated IL-15 peptides contemplated by the present disclosure. By way of example, parameters such as the EC50, the maximal activation, and the immunogenicity of the pegylated IL-15 peptides may be important in determining whether they are viable therapeutic candidates. In some embodiments, one or more parameters of a pegylated IL-15 peptide may be less favorable than those parameters in wild-type IL-15, but the parameters of the pegylated IL-15 peptide taken as a whole result in the peptide being a viable therapeutic candidate.

Bioactivity may be determined by any method known in the art, including a chemokine release assay, a TNFα production assay, a CTLL-2 cell proliferation assay, a MO7e cell proliferation assay, or a T-cell IFNγ secretion assay. The T-cell screening can be performed using CD4+cells, CD8+cells, or NK cells. The skilled artisan is familiar with such assays, and exemplary protocols for several of them are described herein. Likewise, the immunogenicity of the pegylated IL-15 peptides may be predicted or determined by any method known to the skilled artisan, including prediction by screening for at least one of T-cell epitopes or B-cell epitopes. In one aspect, immunogenicity is predicted by an in silico system and/or in an ex vivo assay system.

The pegylated IL-15 peptides contemplated herein may comprise at least one PEG molecule covalently attached through a linker to at least one amino acid residue of IL-15 (e.g., N-terminal or C-terminal pegylation). Linkers are described in detail hereafter. In some embodiments, two or more different sites on IL-15 may be pegylated by introducing more than one mutation and then modifying each of them. In further embodiments, the N-terminus may be pegylated in combination with the introduction of one or more mutations, and the pegylation thereof, elsewhere within the IL-15 protein. In still further embodiments, the C-terminus may be pegylated in combination with the introduction of one or more mutations, and the pegylation thereof, elsewhere within the IL-15 protein. Tyrosine 26 of IL-15 might be pegylated in combination with pegylation of the N-terminus. In additional embodiments, an IL-15 peptide may comprise pegylation at the N-terminus and the C-terminus. Exemplary pegylation conditions are known to the skilled artisan. In further embodiments, the N-terminus may be pegylated in combination with the introduction of one or more mutations, and the pegylation thereof, elsewhere within the IL-15 protein. The PEG component may be any PEG tolerated by the peptides.

Because of the relatively small size of IL-15, the molecular mass of the PEG may be larger than that used for many other protein therapeutics. By way of example, the PEG component of the modified peptide has a molecular mass from 5 kDa to 20 kD in some embodiments, a molecular mass greater than 20 kDa in other embodiments, a molecular mass greater than 25 kDa in certain embodiments, a molecular mass greater than 30 kDa in still other embodiments, a molecular mass greater than 35 kDa in further embodiments, or a molecular mass of at least 40 kD in still other embodiments. In particular embodiments, the PEG has a molecular mass between 20 and 40 kDa. PEGs having other molecular mass values are described herein.

Particular embodiments of the present disclosure comprise a multi-arm PEG IL-15 molecule having the formula:

wherein x, w and z represent components of a PEG, and the IL-15 is covalently attached, optionally via a linker, to w. Embodiments are contemplated wherein the MW of each of x, w and z is the same, the MW of at least one of x, w and z is different, the MW of each of x and z is the same, and wherein the MW of each of x and z is different. The present disclosure contemplates embodiments wherein the MW of the PEG is from 7.5 kDa to 80 kDa, is from 15 kDa to 45 kDa, is from 15 kDa to 60 kDa, is from 15 kDa to 80 kDa, is from 20 kDa to 30 kDa, is from 20 kDa to 40 kDa, is from 20 kDa to 60 kDa, is from 20 kDa to 80 kDa, is from 30 kDa to 40 kDa, is from 30 kDa to 50 kDa, is from 30 kDa to 60 kDa, is from 30 kDa to 80 kDa, is from 40 kDa to 60 kDa, or is from 40 kDa to 80 kDa. In particular embodiments, the MW of each of x and z is 20 kDa, and the MW of w is 10 kDa. Other sizes of PEG, PEG distributions, and the like are described hereafter and are contemplated herein.

In further particular embodiments, the present disclosure contemplates a branched PEG IL-15 molecule having the formula:

wherein x and z represent components of a PEG, and the IL-15 is covalently attached to the PEG via a linker w. In certain embodiments, the MW of the PEG is about 20 kDa, about 30 kDa, about 40 kDa, about 50 kDa, about 60 kDa, about 70 kDa, or about 80 kDa or more. Particular embodiments are contemplated wherein the MW of each of x and z is 10 kDa, 20 kDa, 30 kDa, or 40 kDa.

The present disclosure contemplates embodiments wherein a PEG IL-15 molecule comprises: a) a Helix A, b) an A/B Inter-helix Junction, c) a Helix B, d) a B/C Inter-helix Junction, e) a Helix C, f) a C/D Inter-helix Junction and g) a Helix D; and wherein the peptide further comprises at least one amino acid substitution comprising: substitution of at least one amino acid residue of Helix A other than amino acid residues 2 (W), 4-12 (NVISDLKKI; SEQ ID NO:7), or 16 (I); or substitution of at least one amino acid residue of the A/B Inter-helix Junction other than amino acid residues 30 (D) or 31 (V); or substitution of at least one amino acid residue of Helix B other than amino acid residues 32 (H), 35 (C), 40 (M), 42-44 (CFL), 47 (L) or 50 (I); or substitution of at least one amino acid residue of the B/C Inter-helix Junction; or substitution of at least one amino acid residue of Helix C other than amino acid residues 59 (I), 61-66 (DTVENL; SEQ ID NO:8), or 68-70 (ILA); or substitution of at least one amino acid residue of the C/D Inter-helix Junction other than amino acid residues 85 (C) or 88 (C); or substitution of at least one amino acid residue of Helix D other than amino acid residues 99 (F), 100 (L), 103 (F), or 105-112 (HIVQMFIN; SEQ ID NO:9). The amino acid substitution(s) is a conservative substitution in certain embodiments.

The present disclosure further contemplates embodiments wherein the PEG IL-15 molecule comprises at least one amino acid substitution at one of the following positions: 1, 3, 13-15, 17-29, 33, 34, 36-39, 41, 45, 48, 49, 51-58, 60, 67, 71-84, 86, 87, 89-98, 101, 102, 104, 113, or 114; embodiments wherein the PEG IL-15 molecule comprises at least one amino acid substitution of a tyrosine for at least one of the amino acid residues at the following positions: 1, 3, 13-15, 17-25, 27-29, 33, 34, 36-39, 41, 45, 48, 49, 51-58, 60, 67, 71-84, 86, 87, 89-98, 101, 102, 104, 113, or 114; and embodiments wherein the PEG IL-15 molecule comprises at least one amino acid substitution of a cysteine for at least one of the amino acid residues at the following positions: 1, 3, 13-15, 17-25, 27-29, 33, 34, 36-39, 45, 48, 49, 51-56, 58, 60, 67, 72-84, 86, 87, 89-98, 101, 102, 104, 113, or 114.

In still further embodiments of the present disclosure, in the PEG IL-15 molecule there is at least one amino acid substitution of an N-X-S glycosylation motif for at least one of the amino acid residues at the following positions: 1, 13-15, 17-22, 27-29, 34, 36, 48, 49, 51-58, 60, 72-82, 84, 87, 89-98, 102, or 104, wherein the asparagine of the N-X-S glycosylation motif represents the amino acid position. In still additional embodiments of the present disclosure, in the PEG IL-15 molecule there is at least one amino acid substitution of an N-X-T glycosylation motif for at least one of the amino acid residues at the following positions: 1, 13-15, 17-22, 29, 34, 36, 48, 49, 51-58, 60, 71-78, 80-82, 84, 87, 89-98, or 102, wherein the asparagine of the N-X-T glycosylation motif represents the amino acid position.

The present disclosure contemplates processes for preparing a PEG IL-15 molecule described herein, comprising the step of reacting IL-15 with an activated PEG linker under conditions in which the linker covalently attaches to one amino acid residue of the IL-15. In particular embodiments, the activated PEG linker is selected from the group consisting of succinimidylcarbonate-PEG, PEG-butyraldehyde, PEG-pentaldehyde, PEG-amido-propionaldehyde, PEG-urethano-propioaldehyde, and PEG-propylaldehyde.

Further embodiments of the present disclosure contemplate a pegylated interleukin-15 molecule comprising the formula: (IL-15-L)ₐ-PEG, wherein a is 2-4 and each L, if present, is a linker covalently attaching the PEG molecule to i) an amino group of a single amino acid residue of each IL-15, wherein the amino group of the single amino acid residue is the alpha amino group of the N-terminal amino acid residue or the epsilon amino group of a lysine amino acid residue, or ii) an N-glycosylation site (e.g., an N-X-S motif or an N-X-T motif). In certain embodiments, a=2, a=3, or a=4.

Additional embodiments of the present disclosure contemplate a PEG-IL-15 molecule comprising at least one branched or multi-arm PEG molecule covalently attached to a single amino acid residue of IL-15, wherein the amino acid residue is i) the alpha amino group of the N-terminal amino acid residue, ii) the epsilon amino group of a lysine amino acid residue, or iii) an N-glycosylation site (e.g., an N-X-S motif or an N-X-T motif); and wherein the PEG is optionally covalently attached to the IL-15 through a linker. In some of these embodiments, the PEG-IL-15 comprises the formula: (PEG)_{b}-L-NH-IL-15, wherein the PEG is a branched polyethylene glycol of molecular weight between 5 kDa and 80 kDa; b is 1-9; and L is an optionally present linker moiety attaching the PEG to the single amino acid residue. In other of these embodiments, the PEG-IL-15 comprises the formula: (PEG)_{b}-L-NH-IL-15, wherein the PEG is a multi-arm polyethylene glycol of molecular weight between 50 kDa and 80 kDa; b is 1-9; and L is an optionally present linker moiety attaching the PEG to the single amino acid residue. In particular embodiments, b is 1 and L is a C₂-C₁₂ alkyl.

The present disclosure includes pharmaceutical compositions comprising the peptides described herein, and a pharmaceutically acceptable diluent, carrier or excipient. In some embodiments, the excipient is an isotonic injection solution. The pharmaceutical compositions may be suitable for administration to a subject (e.g., a human), and may comprise one or more additional prophylactic or therapeutic agents. In certain embodiments, the pharmaceutical compositions are contained in a sterile container (e.g., a single- or multi-use vial or a syringe). A kit may contain the sterile container(s), and the kit may also contain one or more additional sterile containers comprising at least one additional prophylactic or therapeutic agent or any other agent that may be used in pharmacological therapy. Examples of such aspects are set forth herein.

Additional embodiments of the present disclosure comprise a method of treating or preventing a disease, disorder or condition in a subject (e.g., a human), comprising administering a therapeutically effective amount of a peptide described herein. In various embodiments of the present disclosure, the disease, disorder or condition is a proliferative disorder, including a cancer or a cancer-related disorder (e.g., a solid tumor or a hematological disorder); an immune or inflammatory disorder (e.g., inflammatory bowel disease, psoriasis, rheumatoid arthritis, sarcoidosis, multiple sclerosis, and Alzheimer's disease); a viral disorder (e.g., human immunodeficiency virus, hepatitis B virus, hepatitis C virus and cytomegalovirus).

In the methods of treating or preventing a disease, disorder or condition, administration of the therapeutically effective amount of a peptide described herein may be by any route appropriate for the peptide, including parenteral injection (e.g., subcutaneously). One or more additional prophylactic or therapeutic agents may be administered with (e.g., prior to, simultaneously with, or subsequent to) the peptide, and/or it may be administered separate from or combined with the peptide.

Additional embodiments will become apparent to the skilled artisan after reviewing the teachings herein.

### BRIEF DESCRIPTION OF THE DRAWINGS

FIG. 1A depicts the IL-15 Long Signal Peptide (LSP) Protein (162 amino acid residues; SEQ ID NO:1). The signal peptide (underlined) comprises residues 1-48.

FIG. 1B depicts the IL-15 Short Signal peptide (SSP) Protein (135 amino acid residues; SEQ ID NO:2). The signal peptide (underlined) comprises residues 1-21.

FIG. 1C depicts the mature human IL-15 protein (114 amino acid residues) (SEQ ID NO:3).

FIG. 2A depicts the Long Signal Peptide (LSP) cDNA Open Reading Frame (ORF) (489 base pairs (SEQ ID NO:4), encoding 162 amino acid residues). The signal peptide (underlined) comprises base pairs 1-144, encoding the first 48 amino acids.

FIG. 2B depicts the Short Signal peptide (SSP) cDNA Open Reading Frame (ORF) (408 base pairs (SEQ ID NO:5), encoding 135 amino acid residues). The signal peptide (underlined) comprises base pairs 1-63, encoding the first 21 amino acids.

FIG. 2C depicts the nucleic acid sequence encoding mature human IL-15 Protein (345 base pairs (SEQ ID NO:6), encoding 114 amino acid residues).

### DETAILED DESCRIPTION

Before the present disclosure is further described, it is to be understood that the disclosure is not limited to the particular embodiments set forth herein, and it is also to be understood that the terminology used herein is for the purpose of describing particular embodiments only, and is not intended to be limiting.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention. Unless defined otherwise, all technical and scientific terms used herein have the same meaning as commonly understood by one of ordinary skill in the art to which this invention belongs.

It must be noted that as used herein and in the appended claims, the singular forms “a,” “an,” and “the” include plural referents unless the context clearly dictates otherwise. It is further noted that the claims may be drafted to exclude any optional element. As such, this statement is intended to serve as antecedent basis for use of such exclusive terminology such as “solely,” “only” and the like in connection with the recitation of claim elements, or use of a “negative” limitation.

The publications discussed herein are provided solely for their disclosure prior to the filing date of the present application. Further, the dates of publication provided may be different from the actual publication dates, which may need to be independently confirmed.

### Overview

The present disclosure contemplates pegylated IL-15 molecules, including pegylated variants, muteins and other IL-15—related molecules as described herein. The skilled artisan will recognize that such molecules may have favorable characteristics and properties, including an extended half-life allowing less frequent dosing. The IL-15 molecules described herein, and compositions (e.g., pharmaceutical compositions) thereof, may be used to treat and/or prevent various diseases, disorders and conditions, and/or the symptoms thereof, including, for example, inflammatory- and immune-related disorders, and cancer and cancer-related disorders.

It should be noted that any reference to “human” in connection with the polypeptides and nucleic acid molecules of the present disclosure is not meant to be limiting with respect to the manner in which the polypeptide or nucleic acid is obtained or the source, but rather is only with reference to the sequence as it may correspond to a sequence of a naturally occurring human polypeptide or nucleic acid molecule. In addition to the human polypeptides and the nucleic acid molecules which encode them, the present disclosure contemplates IL-15—related polypeptides and corresponding nucleic acid molecules from other species.

### Definitions

Unless otherwise indicated, the following terms are intended to have the meaning set forth below. Other terms are defined elsewhere throughout the specification.

The terms “patient” or “subject” are used interchangeably to refer to a human or a non-human animal (e.g., a mammal).

The terms “administration”, “administer” and the like, as they apply to, for example, a subject, cell, tissue, organ, or biological fluid, refer to contact of, for example, a pegylated IL-15, α nucleic acid encoding an IL-15 molecule that may then be pegylated, a pharmaceutical composition comprising the foregoing, or a diagnostic agent; to the subject, cell, tissue, organ, or biological fluid. In the context of a cell, administration includes contact (e.g., in vitro or ex vivo) of a reagent to the cell, as well as contact of a reagent to a fluid, where the fluid is in contact with the cell.

The terms “treat”, “treating”, treatment” and the like refer to a course of action (such as administering a pegylated IL-15 or a pharmaceutical composition comprising a pegylated IL-15) initiated after a disease, disorder or condition, or a symptom thereof, has been diagnosed, observed, and the like so as to eliminate, reduce, suppress, mitigate, or ameliorate, either temporarily or permanently, at least one of the underlying causes of a disease, disorder, or condition afflicting a subject, or at least one of the symptoms associated with a disease, disorder, or condition afflicting a subject. Thus, treatment includes inhibiting (e.g., arresting the development or further development of the disease, disorder or condition or clinical symptoms associated therewith) an active disease. The terms may also be used in other contexts, such as situations where a PEG-IL-15 contacts an IL-15 receptor in, for example, the fluid phase or colloidal phase.

The term “in need of treatment” as used herein refers to a judgment made by a physician or other caregiver that a subject requires or will benefit from treatment. This judgment is made based on a variety of factors that are in the realm of the physician's or caregiver's expertise.

The terms “prevent”, “preventing”, “prevention” and the like refer to a course of action (such as administering a pegylated IL-15 or a pharmaceutical composition comprising a pegylated IL-15) initiated in a manner (e.g., prior to the onset of a disease, disorder, condition or symptom thereof) so as to prevent, suppress, inhibit or reduce, either temporarily or permanently, a subject's risk of developing a disease, disorder, condition or the like (as determined by, for example, the absence of clinical symptoms) or delaying the onset thereof, generally in the context of a subject predisposed to having a particular disease, disorder or condition. In certain instances, the terms also refer to slowing the progression of the disease, disorder or condition or inhibiting progression thereof to a harmful or otherwise undesired state.

The term “in need of prevention” as used herein refers to a judgment made by a physician or other caregiver that a subject requires or will benefit from preventative care. This judgment is made based on a variety of factors that are in the realm of a physician's or caregiver's expertise.

The phrase “therapeutically effective amount” refers to the administration of an agent to a subject, either alone or as part of a pharmaceutical composition and either in a single dose or as part of a series of doses, in an amount capable of having any detectable, positive effect on any symptom, aspect, or characteristic of a disease, disorder or condition when administered to the subject. The therapeutically effective amount can be ascertained by measuring relevant physiological effects, and it can be adjusted in connection with the dosing regimen and diagnostic analysis of the subject's condition, and the like. By way of example, measurement of the amount of inflammatory cytokines produced following administration may be indicative of whether a therapeutically effective amount has been used.

The phrase “in a sufficient amount to effect a change” means that there is a detectable difference between a level of an indicator measured before (e.g., a baseline level) and after administration of a particular therapy. Indicators include any objective parameter (e.g., serum concentration of IL-15) or subjective parameter (e.g., a subject's feeling of well-being).

The term “small molecules” refers to chemical compounds having a molecular weight that is less than about 10 kDa, less than about 2 kDa, or less than about 1 kDa. Small molecules include, but are not limited to, inorganic molecules, organic molecules, organic molecules containing an inorganic component, molecules comprising a radioactive atom, and synthetic molecules. Therapeutically, a small molecule may be more permeable to cells, less susceptible to degradation, and less likely to elicit an immune response than large molecules.

The term “ligand” refers to, for example, a peptide, a polypeptide, a membrane-associated or membrane-bound molecule, or a complex thereof, that can act as an agonist or antagonist of a receptor. “Ligand” encompasses natural and synthetic ligands, e.g., cytokines, cytokine variants, analogs, muteins, and binding compositions derived from antibodies, as well as, e.g., peptide mimetics of cytokines and peptide mimetics of antibodies. The term also encompasses an agent that is neither an agonist nor antagonist, but that can bind to a receptor without significantly influencing its biological properties, e.g., signaling or adhesion. Moreover, the term includes a membrane-bound ligand that has been changed, e.g., by chemical or recombinant methods, to a soluble version of the membrane-bound ligand. A ligand or receptor may be entirely intracellular, that is, it may reside in the cytosol, nucleus, or some other intracellular compartment. The complex of a ligand and receptor is termed a “ligand-receptor complex.”

The terms “inhibitors” and “antagonists”, or “activators” and “agonists” refer to inhibitory or activating molecules, respectively, for example, for the activation of, e.g., a ligand, receptor, cofactor, gene, cell, tissue, or organ. Inhibitors are molecules that decrease, block, prevent, delay activation, inactivate, desensitize, or down-regulate, e.g., a gene, protein, ligand, receptor, or cell. Activators are molecules that increase, activate, facilitate, enhance activation, sensitize, or up-regulate, e.g., a gene, protein, ligand, receptor, or cell. An inhibitor may also be defined as a molecule that reduces, blocks, or inactivates a constitutive activity. An “agonist” is a molecule that interacts with a target to cause or promote an increase in the activation of the target. An “antagonist” is a molecule that opposes the action(s) of an agonist. An antagonist prevents, reduces, inhibits, or neutralizes the activity of an agonist, and an antagonist can also prevent, inhibit, or reduce constitutive activity of a target, e.g., a target receptor, even where there is no identified agonist.

The terms “modulate”, “modulation” and the like refer to the ability of a molecule (e.g., an activator or an inhibitor) to increase or decrease the function or activity of an IL-15 molecule (or the nucleic acid molecules encoding them), either directly or indirectly; or to enhance the ability of a molecule to produce an effect comparable to that of an IL-15 molecule. The term “modulator” is meant to refer broadly to molecules that can effect the activities described above. By way of example, a modulator of, e.g., a gene, a receptor, a ligand, or a cell, is a molecule that alters an activity of the gene, receptor, ligand, or cell, where activity can be activated, inhibited, or altered in its regulatory properties. A modulator may act alone, or it may use a cofactor, e.g., a protein, metal ion, or small molecule. The term “modulator” includes agents that operate through the same mechanism of action as IL-15 (i.e., agents that modulate the same signaling pathway as IL-15 in a manner analogous thereto) and are capable of eliciting a biological response comparable to (or greater than) that of IL-15.

Examples of modulators include small molecule compounds and other bioorganic molecules. Numerous libraries of small molecule compounds (e.g., combinatorial libraries) are commercially available and can serve as a starting point for identifying a modulator. The skilled artisan is able to develop one or more assays (e.g., biochemical or cell-based assays) in which such compound libraries can be screened in order to identify one or more compounds having the desired properties; thereafter, the skilled medicinal chemist is able to optimize such one or more compounds by, for example, synthesizing and evaluating analogs and derivatives thereof. Synthetic and/or molecular modeling studies can also be utilized in the identification of the molecules described above.

The “activity” of a molecule may describe or refer to the binding of the molecule to a ligand or to a receptor; to catalytic activity; to the ability to stimulate gene expression or cell signaling, differentiation, or maturation; to antigenic activity; to the modulation of activities of other molecules; and the like. The term may also refer to activity in modulating or maintaining cell-to-cell interactions (e.g., adhesion), or activity in maintaining a structure of a cell (e.g., a cell membrane). “Activity” can also mean specific activity, e.g., [catalytic activity]/[mg protein], or [immunological activity]/[mg protein], concentration in a biological compartment, or the like. The term “proliferative activity” encompasses an activity that promotes, that is necessary for, or that is specifically associated with, for example, normal cell division, as well as cancer, tumors, dysplasia, cell transformation, metastasis, and angiogenesis.

As used herein, “comparable”, “comparable activity”, “activity comparable to”, “comparable effect”, “effect comparable to”, and the like are relative terms that can be viewed quantitatively and/or qualitatively. The meaning of the terms is frequently dependent on the context in which they are used. By way of example, two agents that both activate a receptor can be viewed as having a comparable effect from a qualitative perspective, but the two agents can be viewed as lacking a comparable effect from a quantitative perspective if one agent is only able to achieve 20% of the activity of the other agent as determined in an art-accepted assay (e.g., a dose-response assay) or in an art-accepted animal model. When comparing one result to another result (e.g., one result to a reference standard), “comparable” frequently (though not always) means that one result deviates from a reference standard by less than 35%, by less than 30%, by less than 25%, by less than 20%, by less than 15%, by less than 10%, by less than 7%, by less than 5%, by less than 4%, by less than 3%, by less than 2%, or by less than 1%. In particular embodiments, one result is comparable to a reference standard if it deviates by less than 15%, by less than 10%, or by less than 5% from the reference standard. By way of example, but not limitation, the activity or effect may refer to efficacy, stability, solubility, or immunogenicity. As previously indicated, the skilled artisan recognizes that use of different methodologies may result in IL-15 that is more or less active—either in apparent activity due to differences in calculating protein concentration or in actual activity—than a hIL-15 reference standard. The skilled artisan will be able to factor in these differences in determining the relative bioactivities of an IL-15 molecule versus hIL-15.

The term “response,” for example, of a cell, tissue, organ, or organism, encompasses a change in biochemical or physiological behavior, e.g., concentration, density, adhesion, or migration within a biological compartment, rate of gene expression, or state of differentiation, where the change is correlated with activation, stimulation, or treatment, or with internal mechanisms such as genetic programming. In certain contexts, the terms “activation”, “stimulation”, and the like refer to cell activation as regulated by internal mechanisms, as well as by external or environmental factors; whereas the terms “inhibition”, “down-regulation” and the like refer to the opposite effects.

The terms “polypeptide,” “peptide,” and “protein”, used interchangeably herein, refer to a polymeric form of amino acids of any length, which can include genetically coded and non-genetically coded amino acids, chemically or biochemically modified or derivatized amino acids, and polypeptides having modified polypeptide backbones. The terms include fusion proteins, including, but not limited to, fusion proteins with a heterologous amino acid sequence, fusion proteins with heterologous and homologous leader sequences, with or without N-terminus methionine residues; immunologically tagged proteins; and the like.

As used herein, the terms “variants” and “homologs” are used interchangeably to refer to amino acid or DNA sequences that are similar to reference amino acid or nucleic acid sequences, respectively. The term encompasses naturally-occurring variants and non-naturally-occurring variants. Naturally-occurring variants include homologs (polypeptides and nucleic acids that differ in amino acid or nucleotide sequence, respectively, from one species to another), and allelic variants (polypeptides and nucleic acids that differ in amino acid or nucleotide sequence, respectively, from one individual to another within a species). Thus, variants and homologs encompass naturally occurring DNA sequences and proteins encoded thereby and their isoforms, as well as splice variants of a protein or gene. The terms also encompass nucleic acid sequences that vary in one or more bases from a naturally-occurring DNA sequence but still translate into an amino acid sequence that corresponds to the naturally-occurring protein due to degeneracy of the genetic code. Non-naturally-occurring variants and homologs include polypeptides and nucleic acids that comprise a change in amino acid or nucleotide sequence, respectively, where the change in sequence is artificially introduced (e.g., muteins); for example, the change is generated in the laboratory by human intervention (“hand of man”). Therefore, non-naturally occurring variants and homologs may also refer to those that differ from the naturally-occurring sequences by one or more conservative substitutions and/or tags and/or conjugates.

The term “muteins” as used herein refers broadly to mutated recombinant proteins. These proteins usually carry single or multiple amino acid substitutions and are frequently derived from cloned genes that have been subjected to site-directed or random mutagenesis, or from completely synthetic genes. Unless otherwise indicated, use of terms such as “mutant of IL-15” refer to IL-15 muteins.

The terms “DNA”, “nucleic acid”, “nucleic acid molecule”, “polynucleotide” and the like are used interchangeably herein to refer to a polymeric form of nucleotides of any length, either deoxyribonucleotides or ribonucleotides, or analogs thereof. Non-limiting examples of polynucleotides include linear and circular nucleic acids, messenger RNA (mRNA), complementary DNA (cDNA), recombinant polynucleotides, vectors, probes, primers and the like.

It will be appreciated that throughout this disclosure reference is made to amino acids according to the single letter or three letter codes. For the reader's convenience, the single and three letter amino acid codes are provided below:

| | | | | | |
|---|---|---|---|---|---|
| G | Glycine | Gly | P | Proline | Pro |
| A | Alanine | Ala | V | Valine | Val |
| L | Leucine | Leu | I | Isoleucine | Ile |
| M | Methionine | Met | C | Cysteine | Cys |
| F | Phenylalanine | Phe | Y | Tyrosine | Tyr |
| W | Tryptophan | Trp | H | Histidine | His |
| K | Lysine | Lys | R | Arginine | Arg |
| Q | Glutamine | Gln | N | Asparagine | Asn |
| E | Glutamic Acid | Glu | D | Aspartic Acid | Asp |
| S | Serine | Ser | T | Threonine | Thr |

As used herein in reference to native human IL-15 or an IL-15 mutein, the terms “modified”, “modification” and the like refer to one or more changes that enhance a desired property of human IL-15 or an IL-15 mutein. Such desired properties include, for example, prolonging the circulation half-life, increasing the stability, reducing the clearance, altering the immunogenicity or allergenicity, and enabling the raising of particular antibodies (e.g., by introduction of unique epitopes) for use in detection assays. As discussed in detail hereafter, modifications to human IL-15 or an IL-15 mutein that may be carried out include, but are not limited to, pegylation (covalent attachment of one or more molecules of polyethylene glycol (PEG), or derivatives thereof); glycosylation (e.g., N-glycosylation), polysialylation and hesylation; albumin fusion; albumin binding through, for example, a conjugated fatty acid chain (acylation); Fc-fusion; and fusion with a PEG mimetic. In some embodiments, linkers are used in such modifications and are described hereafter. In particular embodiments of the present disclosure, a modified IL-15 molecule is a pegylated IL-15.

As used herein in the context of the structure of a polypeptide, “N-terminus” (or “amino terminus”) and “C-terminus” (or “carboxyl terminus”) refer to the extreme amino and carboxyl ends of the polypeptide, respectively, while the terms “N-terminal” and “C-terminal” refer to relative positions in the amino acid sequence of the polypeptide toward the N-terminus and the C-terminus, respectively, and can include the residues at the N-terminus and C-terminus, respectively. “Immediately N-terminal” or “immediately C-terminal” refers to a position of a first amino acid residue relative to a second amino acid residue where the first and second amino acid residues are covalently bound to provide a contiguous amino acid sequence.

“Derived from”, in the context of an amino acid sequence or polynucleotide sequence (e.g., an amino acid sequence “derived from” an IL-15 polypeptide), is meant to indicate that the polypeptide or nucleic acid has a sequence that is based on that of a reference polypeptide or nucleic acid (e.g., a naturally occurring IL-15 polypeptide or an IL-15-encoding nucleic acid), and is not meant to be limiting as to the source or method in which the protein or nucleic acid is made. By way of example, the term “derived from” includes homologs or variants of reference amino acid or DNA sequences.

In the context of a polypeptide, the term “isolated” refers to a polypeptide of interest that, if naturally occurring, is in an environment different from that in which it may naturally occur. “Isolated” is meant to include polypeptides that are within samples that are substantially enriched for the polypeptide of interest and/or in which the polypeptide of interest is partially or substantially purified. Where the polypeptide is not naturally occurring, “isolated” indicates that the polypeptide has been separated from an environment in which it was made by either synthetic or recombinant means.

“Enriched” means that a sample is non-naturally manipulated (e.g., by a scientist) so that a polypeptide of interest is present in a) a greater concentration (e.g., at least 3-fold greater, at least 4-fold greater, at least 8-fold greater, at least 64-fold greater, or more) than the concentration of the polypeptide in the starting sample, such as a biological sample (e.g., a sample in which the polypeptide naturally occurs or in which it is present after administration), or b) a concentration greater than the environment in which the polypeptide was made (e.g., as in a bacterial cell).

“Substantially pure” indicates that a component (e.g., a polypeptide) makes up greater than about 50% of the total content of the composition, and typically greater than about 60% of the total polypeptide content. More typically, “substantially pure” refers to compositions in which at least 75%, at least 85%, at least 90% or more of the total composition is the component of interest. In some cases, the polypeptide will make up greater than about 90%, or greater than about 95% of the total content of the composition.

The terms “specifically binds” or “selectively binds”, when referring to a ligand/receptor, antibody/antigen, or other binding pair, indicates a binding reaction which is determinative of the presence of the protein in a heterogeneous population of proteins and other biologics. Thus, under designated conditions, a specified ligand binds to a particular receptor and does not bind in a significant amount to other proteins present in the sample. The antibody, or binding composition derived from the antigen-binding site of an antibody, of the contemplated method binds to its antigen, or a variant or mutein thereof, with an affinity that is at least 2-times greater, at least 10-times greater, at least 20-times greater, or at least 100-times greater than the affinity with any other antibody, or binding composition derived therefrom. In a particular embodiment, the antibody will have an affinity that is greater than about 10⁹ liters/mol, as determined by, e.g., Scatchard analysis (Munsen, et al. 1980 Analyt. Biochem. 107:220-239). IL-15

IL-15, also referred to as MGC9721, is predicted to be 12.8 kDa monomeric glycoprotein encoded by the 34 kb region on chromosome 4q31. IL-15 belongs to the four cc-helix bundle family, other members of which include IL-2, IL-4, IL-7, IL-9, granulocyte colony-stimulating factor (G-CSF), and granulocyte-macrophage colony-stimulating factor (GM-CSF). The genomic structure of human IL-15 contains 9 exons (1-8 and 4A) and eight introns. Humans and mice share a similar intron/exon structure. The overall intron/exon structure of the portion of the IL-15 gene encoding the mature protein is similar to that of the IL-2 gene and other 4 α-helix bundle cytokines.

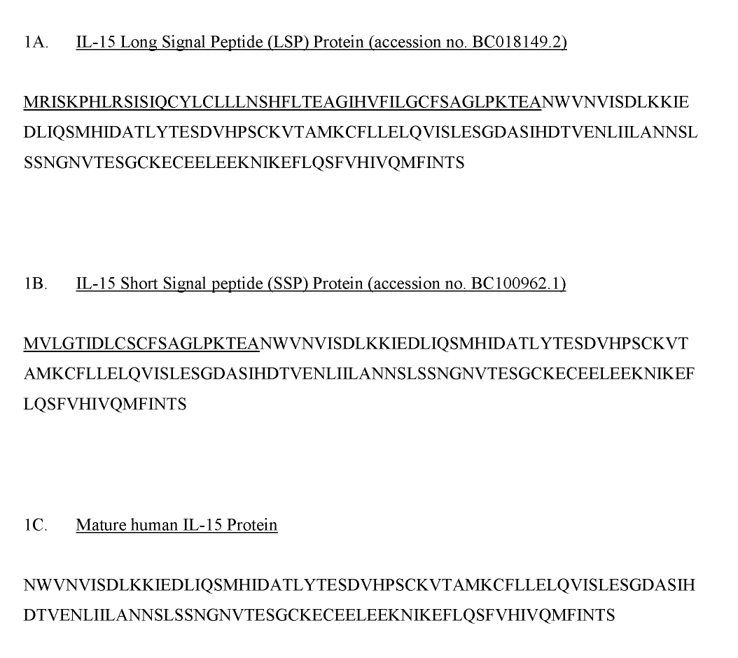

Those of skill in the art will appreciate that IL-15 nucleic acid and amino acid sequences are publicly available in gene databases (e.g., GenBank). As depicted in FIG. 1C (SEQ ID NO:3), the mature human IL-15 protein comprises 114 amino acid residues (12.8 kDa). The recombinant human IL-15 produced in E. coli is a single, non-glycosylated polypeptide chain (115 amino acid residues, including an N-terminal methionine, having a molecular mass of 12.9 kDa). Two transcripts have been reported, both reportedly producing the same mature protein. Referring to FIG. 1A (SEQ ID NO:1), the IL-15 Long Signal Peptide (LSP) Protein (accession no. BC018149.2) comprises 162 amino acid residues, including a 48 residue signal peptide (underlined). Referring to FIG. 1B (SEQ ID NO:2), the IL-15 Short Signal peptide (SSP) Protein (accession no. BC100962.1) comprises 135 amino acid residues, including a 21 residue signal peptide (underlined). The LSP has been described as a secreted protein, and the SSP has been described as remaining intracellular.

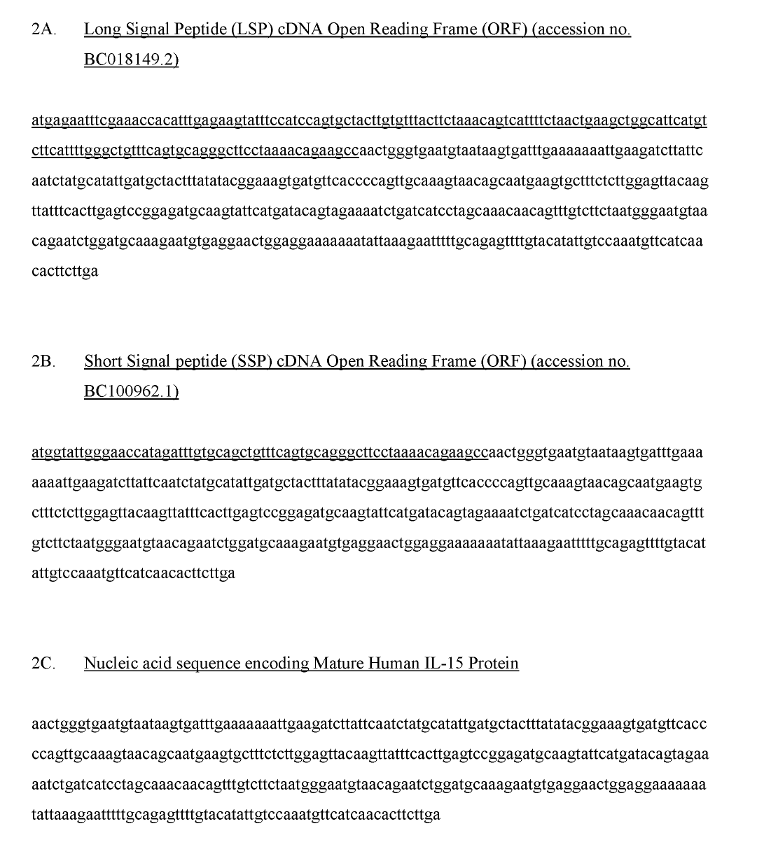

FIG. 2A depicts the Long Signal Peptide (LSP) cDNA ORF (489 base pairs (SEQ ID NO:4), encoding 162 amino acid residues) (accession no. BC018149.2); the signal peptide (underlined) comprises base pairs 1-144, encoding the first 48 amino acids. FIG. 2B depicts the Short Signal peptide (SSP) cDNA ORF (408 base pairs (SEQ ID NO:5), encoding 135 amino acid residues) (accession no. BC100962.1); the signal peptide (underlined) comprises base pairs 1-63, encoding the first 21 amino acids. FIG. 2C depicts the nucleic acid sequence encoding mature human IL-15 Protein (345 base pairs (SEQ ID NO:6), encoding 114 amino acid residues).

Non-human exemplified mammalian IL-15 nucleic acid or amino acid sequences can be from, for example, primate, canine, feline, porcine, equine, bovine, ovine, rodentia, murine, rat, hamster, and guinea pig. Accession numbers for exemplified non-human mammalian IL-15 nucleic acid sequences include U19843 (macaque); DQ021912 (macaque); AB000555 (macaque); NM_214390 (porcine); DQ152967 (ovine); NM_174090 (bovine); NM_008357 (murine); NM_013129 (rattus); DQ083522 (water buffalo); XM_844053 (canine); DQ157452 (lagomorpha); and NM_001009207 (feline). Accession numbers for exemplified non-human mammalian IL-15 amino acid sequences include AAB60398 (macaque); AAY45895 (macaque); NP_999555 (porcine); NP_776515 (bovine); AAY83832 (water buffalo); ABB02300 (ovine); XP 849146 (canine); NP_001009207 (feline); NP_037261 (rattus); and NP_032383 (murine). The identity of mature cynomolygous monkey IL-15 (“cIL-15”) compared to human IL-15 (“hIL-15”) is 96%, while the identity of mature mouse IL-15 (“mIL-15”) and mature hIL-15 is 75%.

Human IL-15 contains two disulfide bonds at positions C42-C88 and C35-C85, the former being homologous to the C-C within IL-2. There are two N-linked glycosylation sites at N79 and N112 (depending on the analytical method used, N71 may be deemed to be a third glycosylation site). The mature IL-15 protein has been predicted to have strong helical moments at amino acid residues 1 to 15, 18 to 57, 65 to 78, and 97 to 114, supporting its 4 α-helix bundle structure (Fehniger, et al., Blood 97(1) (Jan. 1, 2001)).

As indicated previously, a nexus exists between IL-15 and IL-2. Based upon complex regulation and differential patterns of IL-15 and IL-15Rα expression, it is likely that the critical in vivo functions of this receptor/ligand pair differ from those of IL-2 and IL-2Rα. IL-15 exhibits several key non-redundant roles, including its importance during natural killer (NK) cell, NK-T cell, and intestinal intraepithelial lymphocyte development and function. As IL-15 reportedly plays a role in autoimmune processes (e.g., rheumatoid arthritis) and malignancies (e.g., T-cell leukemia), disruptions in normal IL-15 function has been implicated in untoward effects in subjects.

Though both signal through the receptor subunit IL-2R13 and the common γ-chain (γ(c)), IL-15 and IL-2 do not share all of the same biological functions. In the structure of the IL-15-IL-15Rα-IL-2Rβ-γ(c) quaternary complex, IL-15 binds to IL-2Rβ and γ(c) in a heterodimer resembling that of the IL-2-IL-2Rα-IL-2Rβ-γ(c) complex. IL-15Rα has been shown to substantially increase the affinity of IL-15 for IL-2R13, which, in turn, is required for IL-15 trans-signaling. IL-15 and IL-2 induce similar signals, and the specificity of IL-2Rα versus IL-15Rα has been shown to determine cellular responsiveness. (See Ring et al., Nat. Immunol. 13(12):1187-95 (Dec. 13, 2012)).

IL-15 exists primarily as a membrane-bound form, although it also exists as a soluble molecule (Jakobisiak, et al., Cytokine Growth Factor Ref 22(2)99-109 (April 2011)), and it is associated with two distinct signaling mechanisms. The primary mechanism is trans-presentation which is mediated by membrane-bound complex IL-15/IL-15Rα. In this signaling mechanism, IL-15 binds to IL-15Rα receptor, with subsequent presentation to surrounding cells having the IL-15Rβγc complex on their cell surface. The second mechanism is cis-presentation, where IL-15 is presented by IL-15Rα to the 15βγc signaling complex on the same cell.

Referring to the primary signaling mechanism, upon binding of IL-15 to the IL-15Rα receptor and subsequent presentation to surrounding cells bearing IL-15Rβγc complex, the IL-150 subunit activates Janus kinase 1 (Jak1) and the γc subunit activates Janus kinase 2 (Jak2), which leads to phosphorylation and activation of signal transducer and activator of transcription 3 (STAT3) and STAT5. Because IL-15 and IL-2 share receptor subunits, they have similar downstream effects, including the induction of B-cell lymphoma (Bcl-2); mitogen-activated protein kinase (MAP) pathway, and the phosphorylation of lymphocyte-activated protein tyrosine kinase (Lck) and spleen tyrosine kinase (Syk), which results in cell proliferation and maturation (Schluns, et al., Int J Biochem Cell Biol 37(8):1567-71 (August 2005)).

In contrast, the IL-15R signaling pathway in mast cells includes Jak2 and STAT5 instead Jak1/3 and STAT3/5. Phosphorylation STATs form transcription factors and activate transcription of appropriate genes. The β chain of IL-15R recruits and also activates protein tyrosine kinases of the Src family including Lck, Fyn and Lyn kinase. The β chain also activates phosphatidylinositol 3-kinase (PI3K) and AKT signaling pathways and induces expression of various transcription factors, including c-Fos, c-Jun, c-Myc and NF-κB (Jakobisiak, et al., Cytokine Growth Factor Ref 22(2)99-109 (April 2011)).

### Pegylated IL-15

The utility of recombinant human IL-15 is frequently limited by its relatively short serum half-life, which may be due to, for example, renal clearance or proteolytic degradation. As a result, various approaches have been explored to improve the pharmacokinetic profile of IL-15 without adversely disrupting its structure and thus having an undesirable impact on activity. Pegylation of IL-15 results in improvement of certain pharmacokinetic parameters (e.g., serum half-life), as reported in, for example, CN102145178.

Pegylation of IL-15 may occur at one or more of the N-terminus, the C-terminus, or internally. In particular embodiments, the present disclosure contemplates pegylation at the N-terminus. As will be apparent to the skilled artisan, more than one polyethylene glycol molecule may be attached to more than one amino acid residue. Thus, as used herein, the terms “pegylated IL-15” and “PEG-IL-15” refer to an IL-15 molecule having one or more polyethylene glycol molecules covalently attached to at least one amino acid residue of the IL-15 protein, generally via a linker, such that the attachment is stable. The terms “monopegylated IL-15” and “mono-PEG-IL-15” may be used to indicate that one polyethylene glycol molecule is covalently attached to a single amino acid residue of IL-15, generally via a linker. The terms “dipegylated IL-15” and “di-PEG-IL-15” may be used to describe an IL-15 protein wherein one polyethylene glycol molecule is covalently attached to one amino acid residue, and another polyethylene glycol molecule is covalently attached to a different amino acid residue. For example, one polyethylene glycol molecule may be covalently bound to the N-terminal amino acid residue of mature IL-15, and another polyethylene glycol molecule may be covalently bound to the C-terminal residue. It is also possible to generate a protein wherein a polyethylene molecule is covalently attached to more than two amino acid residues; one of ordinary skill in the art is familiar with means of producing such molecules.

In particular embodiments, the PEG-IL-15 used in the present disclosure is a mono-PEG-IL-15 in which one to nine PEG molecules are covalently attached via a linker to the alpha amino group of the amino acid residue at the N-terminus or the epsilon amino group on the side chain of lysine residues. Linkers are described further hereafter. In order to effect pegylation at sites within mature IL-15 that might not normally be amenable to pegylation, one or more different sites on IL-15 might be modified by introducing more than one mutation and then modifying (i.e., pegylating) each of them. Exemplary pegylation conditions are described elsewhere herein.

In particular embodiments, the average molecular weight of the PEG moiety is between about 5 kDa and about 80 kDa. For example, the PEG moiety may have a molecular mass greater than about 5 kDa, greater than about 10 kDa, greater than about 15 kDa, greater than about 20 kDa, greater than about 25 kDa, greater than about 30 kDa, greater than about 35 kDa, greater than about 40 kDa, greater than about 45 kDa, greater than about 50 kDa, greater than about 55 kDa, greater than about 60 kDa, greater than about 65 kDa, greater than about 70 kDa, greater than about 75 kDa, or greater than about 80 kDa. In some embodiments, the molecular mass is from about 5 kDa to about 10 kDa, from about 5 kDa to about 15 kDa, from about 5 kDa to about 20 kDa, from about 10 kDa to about 15 kDa, from about 10 kDa to about 20 kDa, from about 10 kDa to about 25 kDa or from about 10 kDa to about 30 kDa. In other embodiments, the molecular mass is from about 15 kDa to about 20 kDa, from about 15 kDa to about 25 kDa, from about 15 kDa to about 30 kDa, from about 15 kDa to about 35 kDa, from about 15 kDa to about 40 kDa, or from about 15 kDa to about 45 kDa.

Because of the size of IL-15, PEGs greater than 20 kDa (e.g., in the 20-40 kDa range) are contemplated in particular embodiments. In some embodiments, the molecular mass is from about 20 kDa to about 25 kDa, from about 20 kDa to about 30 kDa, from about 20 kDa to about 35 kDa, from about 20 kDa to about 40 kDa, from about 20 kDa to about 45 kDa, or from about 20 kDa to about 50 kDa. In some additional embodiments, the molecular mass is from about 25 kDa to about 30 kDa, from about 25 kDa to about 35 kDa, from about 25 kDa to about 40 kDa, from about 25 kDa to about 45 kDa, or from about 25 kDa to about 50 kDa. In still other embodiments, the molecular mass is from about 30 kDa to about 35 kDa, from about 30 kDa to about 40 kDa, from about 30 kDa to about 45 kDa, or from about 30 kDa to about 50 kDa. In further embodiments, the molecular mass is from about 35 kDa to about 40 kDa, from about 35 kDa to about 45 kDa, from about 35 kDa to about 50 kDa, from about 40 kDa to about 45 kDa, from about 40 kDa to about 50 kDa, or from about 45 kDa to about 50 kDa. In still further embodiments, the molecular mass is from about 50 kDa to about 60 kDa, from about 50 kDa to about 70 kDa, from about 50 kDa to about 80 kDa, from about 60 kDa to about 70 kDa, from about 60 kDa to about 80 kDa, or from about 70 kDa to about 80 kDa. The present disclosure contemplates PEGs having molecular masses greater than 80 kDa in 5 kDa increments (e.g., 85 kDa, 90 kDa, 95 kDa, etc.).

Although the present disclosure does not require use of a specific method or site of PEG attachment to IL-15, it is frequently advantageous that pegylation improves, does not alter, or only nominally decreases the activity of the IL-15 molecule. In certain embodiments, the impact of any increase in half-life is greater than the impact of any decrease in biological activity. The biological activity of PEG-IL-15 is frequently measured by assessing the levels of inflammatory cytokines (e.g., IFN-γ) in the serum of subjects challenged with a bacterial antigen (lipopolysaccharide (LPS)) and treated with PEG-IL-15. Other means for measuring bioactivity are described elsewhere herein.

A comprehensive discussion of particular pegylated IL-15 molecules contemplated by the present disclosure is set forth herein.

IL-15 Variants

IL-15 variants can be prepared with various objectives in mind, including increasing serum half-life, reducing an immune response against IL-15, facilitating purification or preparation, decreasing degradation, improving therapeutic efficacy, and lessening the severity or occurrence of side effects during therapeutic use. The amino acid sequence variants are usually predetermined variants not found in nature, although some may be post-translational variants, e.g., glycosylated variants. Any variant of IL-15 can be used provided it retains a suitable level of IL-15 activity. IL-15 activities are described elsewhere herein (e.g., regulation of T cell and natural killer (NK) cell activation and proliferation).

The phrase “conservative amino acid substitution” refers to substitutions that preserve the activity of the protein by replacing an amino acid(s) in the protein with an amino acid with a side chain of similar acidity, basicity, charge, polarity, or size of the side chain. Conservative amino acid substitutions generally entail substitution of amino acid residues within the following groups: 1) L, I, M, V, F; 2) R, K; 3) F, Y, H, W, R; 4) G, A, T, S; 5) Q, N; and 6) D, E. Guidance for substitutions, insertions, or deletions may be based on alignments of amino acid sequences of different variant proteins or proteins from different species. Thus, in addition to any naturally-occurring IL-15 polypeptide, the present disclosure contemplates having 1, 2, 3, 4, 5, 6, 7, 8, 9, or 10 usually no more than 20, 10, or 5 amino acid substitutions, where the substitution is usually a conservative amino acid substitution. If should be noted that one or more unnatural amino acids may be introduced into IL-15 as a means of fostering site-specific conjugation.

The present disclosure also contemplates active fragments (e.g., subsequences) of mature IL-15 containing contiguous amino acid residues derived from the mature IL-15. The length of contiguous amino acid residues of a peptide or a polypeptide subsequence varies depending on the specific naturally-occurring amino acid sequence from which the subsequence is derived. In general, peptides and polypeptides may be from about 20 amino acids to about 40 amino acids, from about 41 amino acids to about 50 amino acids, from about 51 amino acids to about 60 amino acids, from about 61 amino acids to about 70 amino acids, from about 71 amino acids to about 80 amino acids, from about 81 amino acids to about 90 amino acids, from about 91 amino acids to about 100 amino acids, from about 101 amino acids to about 105 amino acids, from about 106 amino acids to about 110 amino acids, or from about 111, 112, or 113 amino acids up to the full-length peptide or polypeptide.

Additionally, IL-15 polypeptides can have a defined sequence identity compared to a reference sequence over a defined length of contiguous amino acids (e.g., a “comparison window”). Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Madison, Wis.), or by manual alignment and visual inspection (see, e.g., Current Protocols in Molecular Biology (Ausubel et al., eds. 1995 supplement)).

As an example, a suitable IL-15 polypeptide can comprise an amino acid sequence having at least about 75%, at least about 80%, at least about 85%, at least about 90%, at least about 95%, at least about 98%, or at least about 99%, amino acid sequence identity to a contiguous stretch of from about 20 amino acids to about 40 amino acids, from about 41 amino acids to about 50 amino acids, from about 51 amino acids to about 60 amino acids, from about 61 amino acids to about 70 amino acids, from about 71 amino acids to about 80 amino acids, from about 81 amino acids to about 90 amino acids, from about 91 amino acids to about 100 amino acids, from about 101 amino acids to about 105 amino acids, from about 106 amino acids to about 110 amino acids, or from about 111, 112, or 113 amino acids up to the full-length peptide or polypeptide.

As discussed further below, the IL-15 polypeptides may be isolated from a natural source (e.g., an environment other than its naturally-occurring environment) and may also be recombinantly made (e.g., in a genetically modified host cell such as bacteria, yeast, Pichia, insect cells, and the like), where the genetically modified host cell is modified with a nucleic acid comprising a nucleotide sequence encoding the polypeptide. The IL-15 polypeptides may also be synthetically produced (e.g., by cell-free chemical synthesis).

Encompassed herein are other IL-15 molecules, including IL-15 fragments; molecules that comprise an IL-15 polypeptide complexed with a heterologous protein; and IL-15 fusion proteins that comprise IL-15 fused, at the nucleic acid level, to one or more therapeutic agents (e.g., an anti-inflammatory biologic). Such molecules may be modified using the approaches described herein or any other approach known to the skilled artisan.

The rational drug design approaches of the present disclosure may utilize crystallographic and similar data from a number of sources. By way of example, the crystal structure of IL-15 in complex with the sushi domain of IL-15Ralpha has been described. Olsen, et al., J. Biol. Chem. 282(51):37191-204 (Dec. 21 2007). In addition, Pettit, et al., J. Biol. Chem. 272:2312-18 (1997)) describe structure-function studies of IL-15 using site-specific mutagenesis, polyethylene glycol conjugation, and homology modeling. Such information and data can be leveraged in the identification and selection of pegylated IL-15 molecules having desirable characteristics.

### Immunogenicity Considerations of Modified Forms of IL-15

Immunogenicity, the ability of an antigen to elicit humoral (B-cell) and/or cell-mediated (T-cell) immune responses in a subject, can be categorized as ‘desirable’ or ‘undesirable’. Desirable immunogenicity typically refers to the subject's immune response mounted against a pathogen (e.g., a virus or bacterium) that is provoked by vaccine injection. In this context, the immune response is advantageous. Conversely, undesirable immunogenicity typically refers to the subject's immune response mounted against an antigen like a therapeutic protein (e.g., IL-15); the immune response can, for example, result in anti-drug-antibodies (ADAs) that adversely impact the therapeutic protein's effectiveness or its pharmacokinetic parameters, and/or contribute to other adverse effects. In this context, the immune response is disadvantageous.

There are a number of subject-specific and product-specific factors that affect a subject's immune reaction to a protein therapeutic. Subject-specific factors include the immunologic status and competence of the subject; prior sensitization/history of allergy; route of administration; dose and frequency of administration; genetic status of the subject; and the subject's status of immune tolerance to endogenous protein. Product-specific factors affecting immunogenicity include product origin (foreign or endogenous); product's primary molecular structure/post-translational modifications, tertiary and quaternary structure, etc.; presence of product aggregates; conjugation/modification (e.g., glycosylation and pegylation); impurities with adjuvant activity; product's immunomodulatory properties; and formulation.

Autologous or human-like polypeptide therapeutics have proven to be surprisingly immunogenic in some applications, and surprisingly non-immunogenic in others. Particular pegylated IL-15 molecules are likely to provoke a range of humoral and cell-mediated immune responses. In certain contexts, conjugation of one or more amino acid residues with a PEG moiety may dramatically reduce the immunogenicity of an otherwise highly immunogenic protein.

### Methods of Production of IL-15

A polypeptide of the present disclosure can be produced by any suitable method, including non-recombinant (e.g., chemical synthesis) and recombinant methods.

Chemical Synthesis

Where a polypeptide is chemically synthesized, the synthesis may proceed via liquid-phase or solid-phase. Solid-phase peptide synthesis (SPPS) allows the incorporation of unnatural amino acids and/or peptide/protein backbone modification. Various forms of SPPS, such as 9-fluorenylmethoxycarbonyl (Fmoc) and t-butyloxycarbonyl (Boc), are available for synthesizing polypeptides of the present disclosure. Details of the chemical syntheses are known in the art (e.g., Ganesan A. (2006) Mini Rev. Med. Chem. 6:3-10; and Camarero J. A. et al., (2005) Protein Pept Lett. 12:723-8).

Solid phase peptide synthesis may be performed as described hereafter. The alpha functions (Nα) and any reactive side chains are protected with acid-labile or base-labile groups. The protective groups are stable under the conditions for linking amide bonds but can readily be cleaved without impairing the peptide chain that has formed. Suitable protective groups for the α-amino function include, but are not limited to, the following: Boc, benzyloxycarbonyl (Z), O-chlorbenzyloxycarbonyl, bi-phenylisopropyloxycarbonyl, tert-amyloxycarbonyl (Amoc), α, α-dimethyl-3,5-dimethoxy-benzyloxycarbonyl, o-nitrosulfenyl, 2-cyano-t-butoxy-carbonyl, Fmoc, 1-(4,4-dimethyl-2,6-dioxocylohex-1-ylidene)ethyl (Dde) and the like.

Suitable side chain protective groups include, but are not limited to: acetyl, allyl (All), allyloxycarbonyl (Alloc), benzyl (Bzl), benzyloxycarbonyl (Z), t-butyloxycarbonyl (Boc), benzyloxymethyl (Bom), o-bromobenzyloxycarbonyl, t-butyl (tBu), t-butyldimethylsilyl, 2-chlorobenzyl, 2-chlorobenzyloxycarbonyl, 2,6-dichlorobenzyl, cyclohexyl, cyclopentyl, dimethyl-2,6-dioxocyclohex-1-ylidene)ethyl (Dde), isopropyl, 4-methoxy-2,3-6-trimethylbenzylsulfonyl (Mtr), 2,3,5,7,8-pentamethylchroman-6-sulfonyl (Pmc), pivalyl, tetrahydropyran-2-yl, tosyl (Tos), 2,4,6-trimethoxybenzyl, trimethylsilyl and trityl (Trt).

In the solid phase synthesis, the C-terminal amino acid is coupled to a suitable support material. Suitable support materials are those which are inert towards the reagents and reaction conditions for the step-wise condensation and cleavage reactions of the synthesis process and which do not dissolve in the reaction media being used. Examples of commercially-available support materials include styrene/divinylbenzene copolymers which have been modified with reactive groups and/or polyethylene glycol; chloromethylated styrene/divinylbenzene copolymers; hydroxymethylated or aminomethylated styrene/divinylbenzene copolymers; and the like.

When preparation of the peptidic acid is desired, polystyrene (1%)-divinylbenzene or TentaGel® derivatized with 4-benzyloxybenzyl-alcohol (Wang-anchor) or 2-chlorotrityl chloride can be used. In the case of the peptide amide, polystyrene (1%) divinylbenzene or TentaGel® derivatized with 5-(4′-aminomethyl)-3′,5′-dimethoxyphenoxy) valeric acid (PAL-anchor) or p-(2,4-dimethoxyphenyl-amino methyl)-phenoxy group (Rink amide anchor) can be used.

The linkage to the polymeric support can be achieved by reacting the C-terminal Fmoc-protected amino acid with the support material by the addition of an activation reagent in ethanol, acetonitrile, N,N-dimethylformamide (DMF), dichloromethane, tetrahydrofuran, N-methylpyrrolidone or similar solvents at room temperature or elevated temperatures (e.g., between 40° C. and 60° C.) and with reaction times of, e.g., 2 to 72 hours.

The coupling of the Na-protected amino acid (e.g., the Fmoc amino acid) to the PAL, Wang or Rink anchor can, for example, be carried out with the aid of coupling reagents such as N,N′-dicyclohexylcarbodiimide (DCC), N,N′-diisopropylcarbodiimide (DIC) or other carbodiimides, 2-(1H-benzotriazol-1-yl)-1,1,3,3-tetramethyluronium tetrafluoroborate (TBTU) or other uronium salts, O-acyl-ureas, benzotriazol-1-yl-tris-pyrrolidino-phosphonium hexafluorophosphate (PyBOP) or other phosphonium salts, N-hydroxysuccinimides, other N-hydroxyimides or oximes in the presence or absence of 1-hydroxybenzotriazole or 1-hydroxy-7-azabenzotriazole, e.g., with the aid of TBTU with addition of HOBt, with or without the addition of a base such as, for example, diisopropylethylamine (DIEA), triethylamine or N-methylmorpholine, e.g., diisopropylethylamine with reaction times of 2 to 72 hours (e.g., 3 hours in a 1.5 to 3-fold excess of the amino acid and the coupling reagents, for example, in a 2-fold excess and at temperatures between about 10° C. and 50° C., for example, 25° C. in a solvent such as dimethylformamide, N-methylpyrrolidone or dichloromethane, e.g., dimethylformamide).

Instead of the coupling reagents, it is also possible to use the active esters (e.g., pentafluorophenyl, p-nitrophenyl or the like), the symmetric anhydride of the Na-Fmoc-amino acid, its acid chloride or acid fluoride, under the conditions described above.

The Na-protected amino acid (e.g., the Fmoc amino acid) can be coupled to the 2-chlorotrityl resin in dichloromethane with the addition of DIEA and having reaction times of 10 to 120 minutes, e.g., 20 minutes, but is not limited to the use of this solvent and this base.

The successive coupling of the protected amino acids can be carried out according to conventional methods in peptide synthesis, typically in an automated peptide synthesizer. After cleavage of the Na-Fmoc protective group of the coupled amino acid on the solid phase by treatment with, e.g., piperidine (10% to 50%) in dimethylformamide for 5 to 20 minutes, e.g., 2×2 minutes with 50% piperidine in DMF and 1×15 minutes with 20% piperidine in DMF, the next protected amino acid in a 3 to 10-fold excess, e.g., in a 10-fold excess, is coupled to the previous amino acid in an inert, non-aqueous, polar solvent such as dichloromethane, DMF or mixtures of the two and at temperatures between about 10° C. and 50° C., e.g., at 25° C. The previously mentioned reagents for coupling the first Na-Fmoc amino acid to the PAL, Wang or Rink anchor are suitable as coupling reagents. Active esters of the protected amino acid, or chlorides or fluorides or symmetric anhydrides thereof, can also be used as an alternative.

At the end of the solid phase synthesis, the peptide is cleaved from the support material while simultaneously cleaving the side chain protecting groups. Cleavage can be carried out with trifluoroacetic acid or other strongly acidic media with addition of 5%-20% V/V of scavengers such as dimethylsulfide, ethylmethylsulfide, thioanisole, thiocresol, m-cresol, anisole ethanedithiol, phenol or water, e.g., 15% v/v dimethylsulfide/ethanedithiol/m-cresol 1:1:1, within 0.5 to 3 hours, e.g., 2 hours. Peptides with fully protected side chains are obtained by cleaving the 2-chlorotrityl anchor with glacial acetic acid/trifluoroethanol/dichloromethane 2:2:6. The protected peptide can be purified by chromatography on silica gel. If the peptide is linked to the solid phase via the Wang anchor and if it is intended to obtain a peptide with a C-terminal alkylamidation, the cleavage can be carried out by aminolysis with an alkylamine or fluoroalkylamine. The aminolysis is carried out at temperatures between about −10° C. and 50° C. (e.g., about 25° C.), and reaction times between about 12 and 24 hours (e.g., about 18 hours). In addition, the peptide can be cleaved from the support by re-esterification, e.g., with methanol.

The acidic solution that is obtained may be admixed with a 3 to 20-fold amount of cold ether or n-hexane, e.g., a 10-fold excess of diethyl ether, in order to precipitate the peptide and hence to separate the scavengers and cleaved protective groups that remain in the ether. A further purification can be carried out by re-precipitating the peptide several times from glacial acetic acid. The precipitate that is obtained can be taken up in water or tert-butanol or mixtures of the two solvents, e.g., a 1:1 mixture of tert-butanol/water, and freeze-dried.

The peptide obtained can be purified by various chromatographic methods, including ion exchange over a weakly basic resin in the acetate form; hydrophobic adsorption chromatography on non-derivatized polystyrene/divinylbenzene copolymers (e.g., Amberlite® XAD); adsorption chromatography on silica gel; ion exchange chromatography, e.g., on carboxymethyl cellulose; distribution chromatography, e.g., on Sephadex® G-25; countercurrent distribution chromatography; or high pressure liquid chromatography (HPLC) e.g., reversed-phase HPLC on octyl or octadecylsilylsilica (ODS) phases.

### Recombinant Production

IL-15 (e.g., murine and human IL-15) can be synthesized in a number of ways using standard techniques known in the art, such as those described herein. IL-15 can be of viral origin, and the cloning and expression of a viral IL-15 from Epstein Barr virus (BCRF1 protein) is disclosed in Moore et al., (1990) Science 248:1230. In addition, recombinant IL-15 is commercially available from a number of sources (e.g., Life Technologies, Grand Island, NY and BioLegend, San Diego, Calif.).

Site-specific mutagenesis (also referred to as site-directed mutagenesis and oligonucleotide-directed mutagenesis) can be used to generate specific mutations in DNA to produce rationally-designed proteins of the present disclosure (e.g., particular IL-15 muteins and other modified versions of IL-15, including domains thereof) having improved or desirable properties. Techniques for site-specific mutagenesis are well known in the art. Early site-specific mutagenesis methods (e.g., Kunkel's method; cassette mutagenesis; PCR site-directed mutagenesis; and whole plasmid mutagenesis, including SPRINP) have been replaced by more precise and efficient methods, such as various in vivo methods that include Delitto perfetto (see Storici F. and Resnick M A, (2006) Methods in Enzymology 409:329-45); transplacement “pop-in pop-out”; direct gene deletion and site-specific mutagenesis with PCR and one recyclable marker; direct gene deletion and site-specific mutagenesis with PCR and one recyclable marker using long homologous regions; and in vivo site-directed mutagenesis with synthetic oligonucleotides (and see, e.g., In Vitro Mutagenesis Protocols (Methods in Molecular Biology), 2nd Ed. ISBN 978-0896039100). In addition, tools for effecting site-specific mutagenesis are commercially available (e.g., Stratagene Corp., La Jolla, Calif.).

Where a polypeptide is produced using recombinant techniques, the polypeptide may be produced as an intracellular protein or as a secreted protein, using any suitable construct and any suitable host cell, which can be a prokaryotic or eukaryotic cell, such as a bacterial (e.g., E. coli) or a yeast host cell, respectively. Other examples of eukaryotic cells that may be used as host cells include insect cells, mammalian cells, and/or plant cells. Where mammalian host cells are used, they may include human cells (e.g., HeLa, 293, H9 and Jurkat cells); mouse cells (e.g., NIH3T3, L cells, and C127 cells); primate cells (e.g., Cos 1, Cos 7 and CV1); and hamster cells (e.g., Chinese hamster ovary (CHO) cells).

A variety of host-vector systems suitable for the expression of a polypeptide may be employed according to standard procedures known in the art. See, e.g., Sambrook et al., 1989 Current Protocols in Molecular Biology Cold Spring Harbor Press, New York; and Ausubel et al. 1995 Current Protocols in Molecular Biology, Eds. Wiley and Sons. Methods for introduction of genetic material into host cells include, for example, transformation, electroporation, conjugation, calcium phosphate methods and the like. The method for transfer can be selected so as to provide for stable expression of the introduced polypeptide-encoding nucleic acid. The polypeptide-encoding nucleic acid can be provided as an inheritable episomal element (e.g., a plasmid) or can be genomically integrated. A variety of appropriate vectors for use in production of a polypeptide of interest are commercially available.

Vectors can provide for extrachromosomal maintenance in a host cell or can provide for integration into the host cell genome. The expression vector provides transcriptional and translational regulatory sequences, and may provide for inducible or constitutive expression where the coding region is operably-linked under the transcriptional control of the transcriptional initiation region, and a transcriptional and translational termination region. In general, the transcriptional and translational regulatory sequences may include, but are not limited to, promoter sequences, ribosomal binding sites, transcriptional start and stop sequences, translational start and stop sequences, and enhancer or activator sequences. Promoters can be either constitutive or inducible, and can be a strong constitutive promoter (e.g., T7).

Expression constructs generally have convenient restriction sites located near the promoter sequence to provide for the insertion of nucleic acid sequences encoding proteins of interest. A selectable marker operative in the expression host may be present to facilitate selection of cells containing the vector. Moreover, the expression construct may include additional elements. For example, the expression vector may have one or two replication systems, thus allowing it to be maintained in organisms, for example, in mammalian or insect cells for expression and in a prokaryotic host for cloning and amplification. In addition, the expression construct may contain a selectable marker gene to allow the selection of transformed host cells. Selectable genes are well known in the art and will vary with the host cell used.

Isolation and purification of a protein can be accomplished according to methods known in the art. For example, a protein can be isolated from a lysate of cells genetically modified to express the protein constitutively and/or upon induction, or from a synthetic reaction mixture by immunoaffinity purification, which generally involves contacting the sample with an anti-protein antibody, washing to remove non-specifically bound material, and eluting the specifically bound protein. The isolated protein can be further purified by dialysis and other methods normally employed in protein purification. In one embodiment, the protein may be isolated using metal chelate chromatography methods. Proteins may contain modifications to facilitate isolation.

The polypeptides may be prepared in substantially pure or isolated form (e.g., free from other polypeptides). The polypeptides can be present in a composition that is enriched for the polypeptide relative to other components that may be present (e.g., other polypeptides or other host cell components). For example, purified polypeptide may be provided such that the polypeptide is present in a composition that is substantially free of other expressed proteins, e.g., less than about 90%, less than about 60%, less than about 50%, less than about 40%, less than about 30%, less than about 20%, less than about 10%, less than about 5%, or less than about 1%.

An IL-15 polypeptide may be generated using recombinant techniques to manipulate different IL-15—related nucleic acids known in the art to provide constructs capable of encoding the IL-15 polypeptide. It will be appreciated that, when provided a particular amino acid sequence, the ordinary skilled artisan will recognize a variety of different nucleic acid molecules encoding such amino acid sequence in view of her background and experience in, for example, molecular biology.

### Amide Bond Substitutions

In some cases, IL-15 includes one or more linkages other than peptide bonds, e.g., at least two adjacent amino acids are joined via a linkage other than an amide bond. For example, in order to reduce or eliminate undesired proteolysis or other means of degradation, and/or to increase serum stability, and/or to restrict or increase conformational flexibility, one or more amide bonds within the backbone of IL-15 can be substituted.

In another example, one or more amide linkages (—CO—NH—) in IL-15 can be replaced with a linkage which is an isostere of an amide linkage, such as —CH₂NH—, —CH₂S—, —CH₂CH₂—, —CH═CH—(cis and trans), —COCH₂—, —CH(OH)CH₂— or —CH₂SO—. One or more amide linkages in IL-15 can also be replaced by, for example, a reduced isostere pseudopeptide bond. See Couder et al. (1993) Int. J. Peptide Protein Res. 41:181-184. Such replacements and how to effect them are known to those of ordinary skill in the art.

### Amino Acid Substitutions

One or more amino acid substitutions can be made in an IL-15 polypeptide. The following are non-limiting examples:

a) substitution of alkyl-substituted hydrophobic amino acids, including alanine, leucine, isoleucine, valine, norleucine, (S)-2-aminobutyric acid, (5)-cyclohexylalanine or other simple alpha-amino acids substituted by an aliphatic side chain from C₁-C₁₀ carbons including branched, cyclic and straight chain alkyl, alkenyl or alkynyl substitutions;

b) substitution of aromatic-substituted hydrophobic amino acids, including phenylalanine, tryptophan, tyrosine, sulfotyrosine, biphenylalanine, 1-naphthylalanine, 2-naphthylalanine, 2-benzothienylalanine, 3-benzothienylalanine, histidine, including amino, alkylamino, dialkylamino, aza, halogenated (fluoro, chloro, bromo, or iodo) or alkoxy (from C₁-C₄)-substituted forms of the above-listed aromatic amino acids, illustrative examples of which are: 2—, 3- or 4-aminophenylalanine, 2—, 3- or 4-chlorophenylalanine, 2-, 3- or 4-methylphenylalanine, 2—, 3- or 4-methoxyphenylalanine, 5-amino-, 5-chloro-, 5-methyl- or 5-methoxytryptophan, 2′-, 3′-, or 4′-amino-, 2′-, 3′-, or 4′-chloro-, 2, 3, or 4-biphenylalanine, 2′-, 3′-, or 4′-methyl-, 2—, 3- or 4-biphenylalanine, and 2- or 3-pyridylalanine;

c) substitution of amino acids containing basic side chains, including arginine, lysine, histidine, ornithine, 2,3-diaminopropionic acid, homoarginine, including alkyl, alkenyl, or aryl-substituted (from C₁-C₁₀ branched, linear, or cyclic) derivatives of the previous amino acids, whether the substituent is on the heteroatoms (such as the alpha nitrogen, or the distal nitrogen or nitrogens, or on the alpha carbon, in the pro-R position for example. Compounds that serve as illustrative examples include: N-epsilon-isopropyl-lysine, 3-(4-tetrahydropyridyl)-glycine, 3-(4-tetrahydropyridyl)-alanine, N,N-gamma, gamma'-diethyl-homoarginine. Included also are compounds such as alpha-methyl-arginine, alpha-methyl-2,3-diaminopropionic acid, alpha-methyl-histidine, alpha-methyl-ornithine where the alkyl group occupies the pro-R position of the alpha-carbon. Also included are the amides formed from alkyl, aromatic, heteroaromatic (where the heteroaromatic group has one or more nitrogens, oxygens or sulfur atoms singly or in combination), carboxylic acids or any of the many well-known activated derivatives such as acid chlorides, active esters, active azolides and related derivatives, and lysine, ornithine, or 2,3-diaminopropionic acid;

d) substitution of acidic amino acids, including aspartic acid, glutamic acid, homoglutamic acid, tyrosine, alkyl, aryl, arylalkyl, and heteroaryl sulfonamides of 2,4-diaminopriopionic acid, ornithine or lysine and tetrazole-substituted alkyl amino acids;

e) substitution of side chain amide residues, including asparagine, glutamine, and alkyl or aromatic substituted derivatives of asparagine or glutamine; and

f) substitution of hydroxyl-containing amino acids, including serine, threonine, homoserine, 2,3-diaminopropionic acid, and alkyl or aromatic substituted derivatives of serine or threonine.

In some cases, IL-15 comprises one or more naturally occurring non-genetically encoded L-amino acids, synthetic L-amino acids, or D-enantiomers of an amino acid. In some embodiments, IL-15 comprises only D-amino acids. For example, an IL-15 polypeptide can comprise one or more of the following residues: hydroxyproline, β-alanine, o-aminobenzoic acid, m-aminobenzoic acid, p-aminobenzoic acid, m-aminomethylbenzoic acid, 2,3-diaminopropionic acid, α-aminoisobutyric acid, N-methylglycine (sarcosine), ornithine, citrulline, t-butylalanine, t-butylglycine, N-methylisoleucine, phenylglycine, cyclohexylalanine, norleucine, naphthylalanine, pyridylalanine 3-benzothienyl alanine, 4-chlorophenylalanine, 2-fluorophenylalanine, 3-fluorophenylalanine, 4-fluorophenylalanine, penicillamine, 1,2,3,4-tetrahydroisoquinoline-3-carboxylic acid, β-2-thienylalanine, methionine sulfoxide, homoarginine, N-acetyl lysine, 2,4-diamino butyric acid, rho-aminophenylalanine, N-methylvaline, homocysteine, homoserine, ϵ-amino hexanoic acid, ω-aminohexanoic acid, ω-aminoheptanoic acid, ω-aminooctanoic acid, ω-aminodecanoic acid, ω-aminotetradecanoic acid, cyclohexylalanine, α,γ-diaminobutyric acid, α,β-diaminopropionic acid, δ-amino valeric acid, and 2,3-diaminobutyric acid.

### Additional Modifications

A cysteine residue or a cysteine analog can be introduced into an IL-15 polypeptide to provide for linkage to another peptide via a disulfide linkage or to provide for cyclization of the IL-15 polypeptide. Methods of introducing a cysteine or cysteine analog are known in the art (see, e.g., U.S. Pat. No. 8,067,532). Other means of cyclization include introduction of an oxime linker or a lanthionine linker; see, e.g., U.S. Pat. No. 8,044,175. Any combination of amino acids (or non-amino acid moieties) that can form a cyclizing bond can be used and/or introduced. A cyclizing bond can be generated with any combination of amino acids (or with an amino acid and —(CH2)—CO— or —(CH2)ₙ—C₆H₄—CO—) with functional groups which allow for the introduction of a bridge. Some examples are disulfides, disulfide mimetics such as the —(CH2)ₙ— carba bridge, thioacetal, thioether bridges (cystathionine or lanthionine) and bridges containing esters and ethers. In these examples, n can be any integer, but is frequently less than ten.

Other modifications include, for example, an N-alkyl (or aryl) substitution (Ψ[CONR]), or backbone crosslinking to construct lactams and other cyclic structures. Other derivatives include C-terminal hydroxymethyl derivatives, o-modified derivatives (e.g., C-terminal hydroxymethyl benzyl ether), N-terminally modified derivatives including substituted amides such as alkylamides and hydrazides.

In some cases, one or more L-amino acids in an IL-15 polypeptide is replaced with one or more D-amino acids.

In some cases, an IL-15 polypeptide is a retroinverso analog (see, e.g., Sela and Zisman (1997) FASEB J. 11:449). Retro-inverso peptide analogs are isomers of linear polypeptides in which the direction of the amino acid sequence is reversed (retro) and the chirality, D- or L-, of one or more amino acids therein is inverted (inverso), e.g., using D-amino acids rather than L-amino acids. [See, e.g., Jameson et al. (1994) Nature 368:744; and Brady et al. (1994) Nature 368:692].

An IL-15 polypeptide can include a “Protein Transduction Domain” (PTD), which refers to a polypeptide, polynucleotide, carbohydrate, or organic or inorganic molecule that facilitates traversing a lipid bilayer, micelle, cell membrane, organelle membrane, or vesicle membrane. A PTD attached to another molecule facilitates the molecule traversing a membrane, for example going from extracellular space to intracellular space, or cytosol to within an organelle. In some embodiments, a PTD is covalently linked to the amino terminus of an IL-15 polypeptide, while in other embodiments, a PTD is covalently linked to the carboxyl terminus of an IL-15 polypeptide. Exemplary protein transduction domains include, but are not limited to, a minimal undecapeptide protein transduction domain (corresponding to residues 47-57 of HIV-1 TAT comprising YGRKKRRQRRR; SEQ ID NO:10); a polyarginine sequence comprising a number of arginine residues sufficient to direct entry into a cell (e.g., 3, 4, 5, 6, 7, 8, 9, 10, or 10-50 arginines); a VP22 domain (Zender et al. (2002) Cancer Gene Ther. 9(6):489-96); a Drosophila Antennapedia protein transduction domain (Noguchi et al. (2003) Diabetes 52(7):1732-1737); a truncated human calcitonin peptide (Trehin et al. (2004) Pharm. Research 21:1248-1256); polylysine (Wender et al. (2000) Proc. Natl. Acad. Sci. USA 97:13003-13008); RRQRRTSKLMKR (SEQ ID NO:11); Transportan GWTLNSAGYLLGKINLKALAALAKKIL (SEQ ID NO:12); KALAWEAKLAKALAKALAKHLAKALAKALKCEA (SEQ ID NO:13); and RQIKIWFQNRRMKWKK (SEQ ID NO:14). Exemplary PTDs include, but are not limited to, YGRKKRRQRRR (SEQ ID NO:10), RKKRRQRRR (SEQ ID NO:15); an arginine homopolymer of from 3 arginine residues to 50 arginine residues; exemplary PTD domain amino acid sequences include, but are not limited to, any of the following: YGRKKRRQRRR (SEQ ID NO:10); RKKRRQRR (SEQ ID NO:16); YARAAARQARA (SEQ ID NO:17); THRLPRRRRRR (SEQ ID NO:18); and GGRRARRRRRR (SEQ ID NO:19).

The carboxyl group COR₃ of the amino acid at the C-terminal end of an IL-15 polypeptide can be present in a free form (R₃=OH) or in the form of a physiologically-tolerated alkaline or alkaline earth salt such as, e.g., a sodium, potassium or calcium salt. The carboxyl group can also be esterified with primary, secondary or tertiary alcohols such as, e.g., methanol, branched or unbranched C₁-C₆-alkyl alcohols, e.g., ethyl alcohol or tert-butanol. The carboxyl group can also be amidated with primary or secondary amines such as ammonia, branched or unbranched C₁-C₆-alkylamines or C₁-C₆ di-alkylamines, e.g., methylamine or dimethylamine.

The amino group of the amino acid NR₁R₂ at the N-terminus of an IL-15 polypeptide can be present in a free form (R₁=H and R₂=H) or in the form of a physiologically-tolerated salt such as, e.g., a chloride or acetate. The amino group can also be acetylated with acids such that R₁=H and R₂=acetyl, trifluoroacetyl, or adamantyl. The amino group can be present in a form protected by amino-protecting groups conventionally used in peptide chemistry, such as those provided above (e.g., Fmoc, Benzyloxy-carbonyl (Z), Boc, and Alloc). The amino group can be N-alkylated in which R₁ and/or R₂=C₁-C₆ alkyl or C₂-C₈ alkenyl or C₇-C₉ aralkyl. Alkyl residues can be straight-chained, branched or cyclic (e.g., ethyl, isopropyl and cyclohexyl, respectively).

Pegylation of IL-15 and Conjugation of IL-15 with other Non-proteinaceous Polymers

PEGs suitable for conjugation to a polypeptide sequence are generally soluble in water at room temperature, and have the general formula R(O—CH₂—CH₂)ₙO—R, where R is hydrogen or a protective group such as an alkyl or an alkanol group, and where n is an integer from 1 to 1000. When R is a protective group, it generally has from 1 to 8 carbons. The PEG conjugated to the polypeptide sequence can be linear or branched. Branched PEG derivatives, “star-PEGs” and multi-armed PEGs are contemplated by the present disclosure. A molecular weight (molecular mass) of the PEG used in the present disclosure is not restricted to any particular range. Certain embodiments have molecular weights between 5 kDa and 20 kDa, while other embodiments have molecular weights between 5 kDa and 10 kDa. Further embodiments describing PEGs having additional molecular weights are described elsewhere herein.

The present disclosure also contemplates compositions of conjugates wherein the PEGs have different n values, and thus the various different PEGs are present in specific ratios. For example, some compositions comprise a mixture of conjugates where n=1, 2, 3 and 4. In some compositions, the percentage of conjugates where n=1 is 18-25%, the percentage of conjugates where n=2 is 50-66%, the percentage of conjugates where n=3 is 12-16%, and the percentage of conjugates where n=4 is up to 5%. Such compositions can be produced by reaction conditions and purification methods know in the art. Exemplary reaction conditions are described throughout the specification. Cation exchange chromatography may be used to separate conjugates, and a fraction is then identified which contains the conjugate having, for example, the desired number of PEGs attached, purified free from unmodified protein sequences and from conjugates having other numbers of PEGs attached.

Pegylation most frequently occurs at the alpha amino group at the N-terminus of the polypeptide, the epsilon amino group on the side chain of lysine residues, and the imidazole group on the side chain of histidine residues. Since most recombinant polypeptides possess a single alpha and a number of epsilon amino and imidazole groups, numerous positional isomers can be generated depending on the linker chemistry. General pegylation strategies known in the art can be applied herein. PEG may be bound to a polypeptide of the present disclosure via a terminal reactive group (a “spacer”) which mediates a bond between the free amino or carboxyl groups of one or more of the polypeptide sequences and polyethylene glycol. The PEG having the spacer which may be bound to the free amino group includes N-hydroxysuccinylimide polyethylene glycol, which may be prepared by activating succinic acid ester of polyethylene glycol with N-hydroxysuccinylimide. Another activated polyethylene glycol which may be bound to a free amino group is 2,4-bis(O-methoxypolyethyleneglycol)-6-chloro-s-triazine, which may be prepared by reacting polyethylene glycol monomethyl ether with cyanuric chloride. The activated polyethylene glycol which is bound to the free carboxyl group includes polyoxyethylenediamine.

Conjugation of one or more of the polypeptide sequences of the present disclosure to PEG having a spacer may be carried out by various conventional methods. For example, the conjugation reaction can be carried out in solution at a pH of from 5 to 10, at temperature from 4° C. to room temperature, for 30 minutes to 20 hours, utilizing a molar ratio of reagent to protein of from 4:1 to 30:1. Reaction conditions may be selected to direct the reaction towards producing predominantly a desired degree of substitution. In general, low temperature, low pH (e.g., pH=5), and short reaction time tend to decrease the number of PEGs attached, whereas high temperature, neutral to high pH (e.g., pH>7), and longer reaction time tend to increase the number of PEGs attached. Various means known in the art may be used to terminate the reaction. In some embodiments the reaction is terminated by acidifying the reaction mixture and freezing at, e.g., −20° C. Pegylation of various molecules is discussed in, for example, U.S. Pat. Nos. 5,252,714; 5,643,575; 5,919,455; 5,932,462; and 5,985,263.

As indicated above, pegylation most frequently occurs at the N-terminus, the side chain of lysine residues, and the imidazole group on the side chain of histidine residues. The usefulness of such pegylation has been enhanced by refinement by, for example, optimization of reaction conditions and improvement of purification processes. More recent residue-specific chemistries have enabled pegylation of arginine, aspartic acid, cysteine, glutamic acid, serine, threonine, and tyrosine, as well as the carboxy-terminus. Some of these amino acid residues can be specifically pegylated, while others are more promiscuous or only result in site-specific pegylation under certain conditions.

Current approaches allowing pegylation of additional amino acid residues include bridging pegylation (disulfide bridges), enzymatic pegylation (glutamines and C-terminus) and glycopegylation (sites of O- and N-glycosylation or the glycans of a glycoprotein), and heterobifunctional pegylation. Further approaches are drawn to pegylation of proteins containing unnatural amino acids, intein fusion proteins for C-terminal pegylation, transglutaminase-mediated pegylation, sortase A-mediated pegylation, and releasable and non-covalent pegylation. In addition, combination of specific pegylation approaches with genetic engineering techniques has enabled the polyethylene glycan polymer to essentially couple at any position on the protein surface due to, for example, substitution of specific amino acid residues in a polypeptide with a natural or unnatural amino acid bearing an orthogonal reactive group. See generally, e.g., Pasut, G. and Veronese, F. M., (2012) J. Controlled Release 161:461-72; Roberts, M. J. et al., (2012) Advanced Drug Delivery Rev. 64:116-27; Jevsevar, S. et al., (2010) Biotechnol. J. 5:113-28; and Yoshioka, Y. (2011) Chem. Central J. 5:25.

The therapeutic value of pegylation molecules is well validated. Clinically used PEG conjugates include the following: OMONTYS (Affymax/Takeda); CIMZIA (Nektar/UCB Pharma); MACUGEN (Pfizer); DOXIL (Ortho Biotech); ADAGEN (mPEG per Adenosine Deaminase; Enzon); ONCASPAR (mPEG-L-Asparaginase; Enzon); MICERA (Continuous Erythropoiesis Receptor Activator or Methoxy Polyethylene Glycol-Epoetin Beta; Roche); PEGASYS (Peginterferon Alfa-2a; Roche); PEG-INTRON (Peginterferon Alfa-2b; Schering-Plough); SOMAVERT (Pegvisomant; Pfizer); NEULASTA (Pegfilgrastim; Amgen); and KRYSTEXXA (Pegloticase; Savient). In addition, a number of PEG low-molecular-weight drug conjugates have entered clinical trials, including PROTHECAN (PEG-Camptothecin; Enzon) and NKTR-102 (PEG-Irinotecan; Nektar).

The present disclosure also contemplates the use of PEG mimetics. Recombinant PEG mimetics have been developed that retain the attributes of PEG (e.g., enhanced serum half-life) while conferring several additional advantageous properties. By way of example, simple polypeptide chains (comprising, for example, Ala, Glu, Gly, Pro, Ser and Thr) capable of forming an extended conformation similar to PEG can be produced recombinantly already fused to the peptide or protein drug of interest (e.g., Amunix' XTEN technology; Mountain View, Calif.). This obviates the need for an additional conjugation step during the manufacturing process. Moreover, established molecular biology techniques enable control of the side chain composition of the polypeptide chains, allowing optimization of immunogenicity and manufacturing properties.

Linkers: Linkers and their use have been described above. Any of the foregoing components and molecules used to modify the polypeptide sequences of the present disclosure may optionally be conjugated via a linker. Suitable linkers include “flexible linkers” which are generally of sufficient length to permit some movement between the modified polypeptide sequences and the linked components and molecules. The linker molecules are generally about 6-50 atoms long. The linker molecules may also be, for example, aryl acetylene, ethylene glycol oligomers containing 2-10 monomer units, diamines, diacids, amino acids, or combinations thereof. Suitable linkers can be readily selected and can be of any suitable length, such as 1 amino acid (e.g., Gly), 2, 3, 4, 5, 6, 7, 8, 9, 10, 10-20, 20-30, 30-50 or more than 50 amino acids.

Exemplary flexible linkers include glycine polymers (G)ₙ, glycine-serine polymers (for example, (GS), GSGGSₙ (SEQ ID NO:20), GGGSₙ (SEQ ID NO:21), (GₘSₒ)ₙ, (GₘSₒGₘ)ₙ, (GₘSₒGₘSₒGₘ)ₙ (SEQ ID NO:22), (GSGGSₘ)ₙ (SEQ ID NO:23), (GSGSₘG)ₙ (SEQ ID NO:24) and (GGGSₘ)ₙ (SEQ ID NO:25), and combinations thereof, where m, n, and o are each independently selected from an integer of at least one), glycine-alanine polymers, alanine-serine polymers, and other flexible linkers. Glycine and glycine-serine polymers are relatively unstructured, and therefore may serve as a neutral tether between components. Exemplary flexible linkers include, but are not limited to GGSG (SEQ ID NO:26), GGSGG (SEQ ID NO:27), GSGSG (SEQ ID NO:22), GSGGG (SEQ ID NO:28), GGGSG (SEQ ID NO:20), and GSSSG (SEQ ID NO:29).

Activated Linkers: In certain embodiments of the present disclosure, PEG is conjugated to IL-15 through an activated linker that is covalently attached to one or more PEG molecules. A linker is “activated” if it is chemically reactive and ready for covalent attachment to a reactive group on a peptide. Activated PEGs comprise a variety of functional groups which enable introduction of the PEG chains into drugs, enzymes, phospholipids and other biologics.

The present disclosure contemplates the use of any activated linker provided that it can accommodate one or more PEG molecules and form a covalent bond with an amino acid residue under suitable reaction conditions. In particular aspects, the activated linker attaches to an alpha amino group in a highly selective manner over other attachment sites (e.g., the epsilon amino group of lysine or the imino group of histidine).

In some embodiments, an activated linker can be represented by the formula: (PEG)_{b}-L′, wherein one or more PEGs are covalently attached to a carbon atom of the linker to form an ether bond, b is 1 to 9 (i.e., 1 to 9 PEG molecules can be attached to the linker), and L′ contains a reactive group (an activated moiety) which can react with, for example, an amino or imino group on an amino acid residue to provide a covalent attachment of the PEG to IL-15. In other embodiments, an activated linker (L′) contains an aldehyde of the formula RCHO, where R is a linear or branched C₁₋₁₁ alkyl; after covalent attachment of an activated linker to IL-15, the linker contains 2 to 12 carbon atoms. The present disclosure contemplates embodiments wherein PEG-propionaldehyde is an exemplary activated linker. PEG-propionaldehyde (CH₂CH₂CHO) is described in U.S. Pat. No. 5,252,714 and is commercially available (e.g., Shearwater Polymers (Huntsville, Ala.). Other activated PEG-linkers can be obtained commercially from, e.g., Shearwater Polymers and Enzon, Inc. (Piscataway, N.J.).

In particular embodiments of the present disclosure, the activated linker is selected from the group consisting of succinimidylcarbonate-PEG, PEG-butyraldehyde, PEG-pentaldehyde, PEG-amido-propionaldehyde, PEG-urethano-propioaldehyde, and PEG-propylaldehyde.

The following sections describe the use of pegylation technology in more detail (and see generally Shashwat, S. et al. (2012) Journal of Drug Delivery Vol. 2012, Article ID 103973 (17 pp.).

Polyethylene Glycol (PEG) and Pegylation of Proteins

Biomolecules can be protected through covalent binding with another molecule, a process referred to as bioconjugation. Many polymers, from both biological and synthetic origins, are used to protect biomolecules. The resulting polymer bioconjugates are characterized by improved properties such as reduced immunogenicity, decreased antibody recognition, increased in vivo residence time, increased drug targeting specificity, and improved pharmacokinetics. Commonly used polymers in drug delivery applications include poly(N-(2-hydroxypropyl) methacrylamide) (PHPMA), poly(oligoethylene glycol methyl ether methacrylate) (POEGMA), poly(D,L-lactic-co-glycolic acid) (PLGA), poly(glutamic acid) (PGA), poly(N-isopropyl acrylamide) (PNIPAM), poly(N,N′- diethyl acrylamide) (PDEAM), polystyrene and poly(ethylene glycol) (PEG).

PEG, the most common abbreviation for polyethylene glycol [poly(ethylene glycol)], refers to a chemical compound composed of repeating ethylene glycol units. Depending on how one chooses to define the constituent monomer or parent molecule (as ethylene glycol, ethylene oxide or oxyethylene), PEG compounds are also known as PEO (polyethylene oxide) and POE (polyoxyethylene). The applications of pegylation can be extended to peptides, enzymes, antibody fragments, nucleotides and small organic molecules. PEG is synthesized by anionic polymerization of ethylene oxide initiated by nucleophilic attack of a hydroxide ion on the epoxide ring. Most useful for polypeptide modification is monomethoxy PEG (mPEG).

PEG is biocompatible, lacks immunogenicity, antigenicity and toxicity, is soluble in water and other organic solvents, is readily cleared from the body and has high mobility in solution, making it the polymer of choice for bioconjugation (see Pasut, G., et al. (2006) Polymer Therapeutics I, 192, 95-134). Successful conjugation of PEG with biomolecule depends upon the chemical structure, molecular weight, steric hindrance, and the reactivity of the biomolecule as well as the polymer. Bioconjugate synthesis requires both chemical entities (i.e., the bioactive as well as the polymer) to possess a reactive or functional group such as —COOH, —OH, —SH, or —NH₂; therefore, the synthetic methodology to form a conjugate involves either protection or deprotection of the groups. The conjugation of a biomolecule with PEG will result in the modification of its physiochemical properties, particularly size, and increase the systemic retention of the therapeutic agent in the body. It may also enable the moiety to cross the cell membrane by endocytosis to reach particular intracellular targets (Khandare, J. and Minko, T. (2006) Progress in Polymer Science 31(4):359-97). Moreover, PEG is one of a small number of synthetic polymers generally regarded as safe by the US FDA for internal administration (see Bhattarai, N. et al. (2005) Macromolecular Bioscience 5(2):107-11).

As noted above, pegylation can impart several significant and distinct pharmacological advantages over the unmodified form, including improved drug solubility; reduced dosage frequency, toxicity and rate of kidney clearance; an extended circulating life, increased drug stability; enhanced protection from proteolytic degradation; decreased immunogenicity and antigenicity; and minimal loss of biological activity (see, e.g., Kozlowski, A. and Harris, J. M. (2001) Journal of Controlled Release 72(1-3):217-24). The reduced kidney clearance of pegylated proteins can be attributed to an apparent shielding of protein surface charges and an increased hydrodynamic volume of the conjugated product due to the ability of PEG molecules to coordinate with two to three water molecules per monomer unit.

In addition to these pharmacological advantages, pegylation can substantially alter the physicochemical properties of the parent protein, including its electrostatic and hydrophobic properties. Pegylation significantly influences the elimination pathway of the molecule, by shifting from a renal to a hepatic pathway. The tissue-organ distribution profile of the molecule is also greatly influenced by pegylation, wherein pegylated proteins preferably follow a peripheral distribution (Hamidi, M. et al. (2006) Drug Delivery 13(6):399-4090).

Protein Conjugation

The pegylation process has developed from non-specific random conjugations, referred to as “first generation PEGylation”, to site-specific conjugation methods referred to as “second generation PEGylation”. The increase in pegylation specificity is primarily attributable to the availability of more specific functionalization of PEG molecules capable of reacting with particular functional moieties in the protein. The result is controlled, well-defined conjugated products with improved product profiles over those obtained through non-specific random conjugations.

Precise and versatile application of PEG in proteomics and other biological research methods depends upon the availability of polyethylene glycol derivatives of defined length (MW) that are activated with specific functional groups. Purified PEG is most commonly available commercially as mixtures of different oligomer sizes in broadly or narrowly defined molecular weight (MW) ranges. For example, “PEG 600” typically refers to a preparation that includes a mixture of oligomers having an average MW of 600 g/mol. Similarly, “PEG 10000” refers to a mixture of PEG molecules (n=195 to 265) having an average MW of 10,000 g/mol.

Amine conjugation. The coupling reactions involving amine groups are usually of two types—acylation or alkylation. Because of the availability of a number of accessible primary amino groups on the surface of a protein, conjugation through this functional group is the most extensively used method. Lysine, ornithine and N-terminal amino groups are the most commonly exploited (see Bruckdorfer, T., (2008, (Spring)) Drug delivery with PEGylaton. European Biopharmaceutical Review 96-104). Early amine conjugation strategies often resulted in non-specific pegylation. The introduction of PEG aldehyde derivatives (e.g., PEG-propionaldehyde) capable of forming a stable secondary amine linkage with amino groups through reductive alkylation using sodium cyanoborohydride resulted in greater specificity and selectivity than previous N-alkyl conjugation strategies. Because the reactivity of aldehyde groups depends on the nucleophilicity of amine groups, reaction will take place only when the pH of the medium is near or above the pKa of that particular amine terminal. Thus, by controlling the pH of the reaction medium, the heterogeneity of the product profile can be greatly reduced. The introduction of monosubstituted propionic and butanoic acid PEG derivatives and their subsequent activation using succinimide derivatives contributed a significant improvement in amine conjugation.

In contrast, the acylation of the N-terminal amino acids results in the formation of stable amide and urethane linkages. PEG derivatives activated with succinimidyl succinate (PEG-SS), succinimidyl carbonate (PEG-SC), benzotriazole carbonate (PEG-BTC), phenyl carbonate, carbonylimidazole and thiazolidine-2-thione have been extensively used in protein conjugation, following the N-terminal acylation pathway. PEG-NETS esters are readily available which are reactive with nucleophiles to release the NETS leaving group and forms an acylated product. NETS is a choice for amine coupling because of its higher reactivity at physiological pH reactions in bioconjugation synthesis. In particular, carboxyl groups activated with NETS esters are highly reactive with amine nucleophiles and are very common entity in peptides and proteins. Polymers containing reactive hydroxyl groups (e.g., PEG) can be modified to obtain anhydride compounds, whereas mPEG can be acetylated with anhydrides to form an ester terminating to free carboxylate groups.

Thiol conjugation. Many coupling methodologies use a heterobifunctional reagent to couple modified lysine residues on one protein to sulfhydryl groups on a second protein, where the modified lysine residues result from the use of a heterobifunctional reagent comprising an NETS functional group, together with a maleimide or protected sulfhydryl group. The linkage formed is either a disulfide bridge or as a thioether bond, depending on whether the introduced group is either a sulfhydryl or maleimide, respectively. The thiol group on the second protein may be an endogenous free sulfhydryl, or chemically introduced by modification of lysine residues.

Selective thiol conjugation with natural or genetically engineered, unpaired cysteine residues provides a site-specific conjugation methodology. Thiol-selective derivatives such as PEG-maleimide, vinylsulfone, iodoacetamide, and orthopyridyl disulfide are used for cysteine conjugation through formation of thioether or disulfide linkages. Examples using PEG-maleimide include those at the genetically introduced cysteine residue of trichosanthin (TCS) using 5 and 20 kDa, antitumor necrosis factor-α-scFv fragment (anti-TNF-α-scFv) using 5, 20 and 40 kDa and recombinant staphylokinase (Sak) using 5, 10 and 20 kDa derivatives. However, because of the limited availability of single cysteine residues and the chances of protein dimerization resulting from the introduction of genetically engineered cysteines, this is not a commonly used strategy. Alternative strategies take advantage of a higher number of accessible disulfide linkages present with paired cysteines in proteins.

Oxidized carbohydrate or N-terminal conjugation. The enzymatic (e.g., glucose oxidase) or chemical (e.g., sodium periodate) oxidation of carbohydrate groups present in glycoproteins or N-terminal serine or threonine residues generates reactive aldehyde groups, which can be further conjugated with PEG hydrazide or amine derivatives. This methodology has been used for PEGylating immunoglobulin G (IgG), which contains nearly 4% carbohydrate, wherein IgG was first oxidized with periodate and then conjugated with mPEG-hydrazide derivative.

Transglutaminase (TGase)—mediated enzymatic conjugation. An alternative conjugation strategy for site-specific PEGylation targets glutamine residues using a TGase-catalyzed acyl transfer reaction between the glutamine (Gln) terminal and PEG primary amino group. TGase-catalyzed selective pegylations of apomyoglobin (apoMb), α-lactalbumin (α-LA), human growth hormone (hGH), human granulocyte colony-stimulating factor (hG-CSF) and human interlukin-2 (hIL-2) with PEG amines have utilized this technique.

Miscellaneous conjugation chemistries. The site-specific process known as GlycoPEGylation uses an enzymatic N-acetylgalactosamine (GalNAc) O-glycolization followed by PEGylation of the introduced O-glycans using a PEG sailic acid derivative. Also, the click chemistry strategies can be used to drive site-specific mono-PEGylation of genetically modified superoxide dismutase (SOD) using a PEG-alkyne derivative to attach to the azide terminal.

Exemplary pegylation conditions. Various means of coupling PEG derivatives to proteins are known to the skilled artisan (see generally Abuchowski, A., et al. (1984) Cancer Biochem. Biophys. 7, 175; Sartore, L., et al. (1991) Appl. Biochem. Biotechnol. 27, 45; and U.S. Pat. No. 5,824,784) and are described elsewhere herein. The following are exemplary conditions, and they should not be construed to limit the conditions that may be employed in conjunction with the present disclosure.

Coupling of PEG-NHS derivatives to protein amines (PEG-NHS +Protein-NH₂)—exemplary conditions no. 1: 50 mM phosphate buffer (pH 7.2), 4° C., 6 hrs; exemplary conditions no. 2: Borate-phosphate buffer (pH 8.0), 25° C., 2 hrs.

Coupling of PEG-Aldehyde derivatives to the NH₂ group of proteins (PEG-aldehyde+Protein-NH₂): sodium cyanoborohydride (10 eq.), 4° C., 20 hrs.

Coupling of PEG-Maleimide derivatives to the SH group of proteins (PEG-Maleimide+Protein-SH): 100 mM phosphate buffer (pH 6.5), 4° C., 4 hrs.

Coupling of PEG-NH₂ derivatives to the COOH group of proteins (PEG-NH₂ Protein—COOH): 50mM phosphate buffer (pH 7.2), WSC (2 eq.), 4° C., 10 hrs.

Coupling of PEG-p-Nitrophenyloxycarbonyl derivatives to the NH₂ group of proteins (PEG-NP+Protein-NH₂): borate-phosphate buffer (pH 8.0-8.3), r.t, overnight.

Reversible PEGylation

In many cases, the improved physicochemical properties of protein pegylation are offset by a substantial reduction in the in vitro protein activity arising from the permanent linkages formed during PEG conjugation. As a result, a reversible (or releasable) pegylation strategy has been developed in which proteins are attached to PEG derivatives through cleavable linkages, which release the protein in vivo at a predetermined kinetic rate. Examples of reversible PEG derivatives that have been used include bicin, oligo-lactic acid ester, succinic ester, disulfide and β-alanine ester linkers (see, e.g., Filpula, D. and Zhao, H. (2008) Advanced Drug Delivery Reviews 60(1):29-49).

Structure of PEGs

A number of commercial entities offer different series of PEG derivatives with various versatile functional groups. For example, NOF America Corp. (White Plains, N.Y.) offers mono-functional linear PEGs comprising highly purified methoxy PEG as the starting material; bi-functional PEGs, which are the most popular derivatives for cross-linking between proteins, enzymes and other pharmaceutical substances; multi-arm PEGs, wherein varied functional groups are attached to the terminals of multi-arm (e.g., 4 and 8 arms) PEGs; branched PEGs, including 2 arm-, 3 arm- and 4-arm branched type-activated PEGs that possess maleimide, aldehyde, amine and activated NHS as the terminal functional groups; heterofunctional PEGs, wherein the use of hetero-type activated PEGs results in different molecules being conjugated onto the end of each of the PEGs; and forked PEGs, which have the advantage of placing two reactive groups at a precise distance apart.

By way of further example, JenKem Technology USA (Plano, Tex.) offers numerous categories of PEGs, including linear NHS PEGs with cleavable linker (e.g., Methoxy PEG Succinimidyl Succinate; Methoxy PEG Succinimidyl Glutarate); linear carbonate PEGs (e.g., Methoxy PEG Succinimidyl Carbonate; Methoxy PEG Nitrophenyl Carbonate); Y-shaped branched NHS PEGs with stable linker; linear monosaccharide NHS PEGs with stable linker (e.g., Galactose PEG NHS Ester; Glucose PEG NHS Ester); linear methoxy NHS PEGs with stable linker (e.g., Methoxy PEG Succinimidyl Carboxymethyl; Methoxy PEG Succinimidyl Butanoate; Methoxy PEG Succinimidyl Hexanoate; Methoxy PEG Succinimidyl Succinamde; Methoxy PEG Succinimidyl Glutaramide); Y-shaped branched carboxy PEGs; linear carboxy PEGs (e.g., Methoxy PEG Carboxyl; Methoxy PEG Hexanoic Acid); homobifunctional PEGs for amine pegylation (e.g., NHS PEG NETS; carboxyl PEG carboxyl); and heterobifunctional PEGs functionalized with carboxyl or NHS.

Any PEG moiety that is commercially available or that can be synthesized by the skilled artisan is contemplated herein. For example, EP1967212 describes a branched PEG derivative having four mPEG branches, with a terminal COOH group available for protein conjugation. This branched PEG derivative has been successfully conjugated with a number of therapeutic proteins, including IFN-α2b, recombinant streptokinase (r-SK), erythropoietin (EPO), granulocyte-colony stimulating factor (G-CSF) and epidermal growth factor (EGF), through NHS activation, and improved pharmacological properties for these products were observed compared with those obtained from two branched-structure of similar molecular mass.

Some embodiments of the present disclosure contemplate branched PEG IL-15 molecules, wherein IL-15 is covalently attached to more than one PEG. Any suitable branched PEG linker that covalently attaches two or more PEG molecules to an amino group on an amino acid residue of IL-15 (e.g., to an alpha amino group at the N-terminus) can be used. In particular embodiments, a branched linker contemplated by the present disclosure contains two or three PEG molecules. By way of example, a branched PEG linker can be a linear or branched aliphatic group that is hydrolytically stable and contains an activated moiety (e.g., an aldehyde group), which reacts with an amino group of an amino acid residue, as described above; the aliphatic group of a branched linker can contain 2 to 12 carbons. In some embodiments, an aliphatic group can be a t-butyl which may contain, for example, three PEG molecules on each of three carbon atoms (i.e., a total of 9 PEG molecules) and a reactive aldehyde moiety on the fourth carbon of the t-butyl.

Further exemplary branched PEG linkers are described in U.S. Pat. Nos. 5,643,575, 5,919,455, 7,052,868, and 5,932,462. The skilled artisan can prepare modifications to branched PEG linkers by, e.g., addition of a reactive aldehyde moiety.

For purposes of the present disclosure, a branched PEG IL-15 molecule may be represented by the following formula, wherein w is a linker covalently attached to more than one PEG:

The present disclosure contemplates branched PEG IL-15 molecules having multiple PEG size distributions, wherein the branched PEG IL-15 molecule is of a therapeutically acceptable MW. In some embodiments, the MW of x is equivalent to the MW of z, and in other embodiments the MW of x and z differ. In branched PEG IL-15 molecules, the total size of the PEG is attributable to the MW of x plus the MW of z, as the MW of the linker is negligible relative to that of x and z. By way of example, for a branched PEG IL-15 molecule comprising a 20 kDa PEG, x and z can each be 10 kDa in some embodiments, and x can be 5 kDa and z can be ˜15 kDa in other embodiments. Examples of linkers and PEGs are described herein.

Other embodiments of the present disclosure contemplate multi-arm PEG IL-15 molecules. In such embodiments, IL-15 is covalently attached, optionally via a linker, to one or more PEG moieties, at least one of which comprises one or more branches. In particular embodiments, a multi-arm PEG IL-15 molecule may be represented by the following formula:

wherein x, w and z represent components of a PEG, and the IL-15 is covalently attached, optionally via a linker, to w. The present disclosure contemplates multi-arm PEG IL-15 molecules having multiple PEG size distributions, wherein the multi-arm PEG IL-15 molecule is of a therapeutically acceptable MW. In some embodiments, the MW of x, w and z are equivalent. In other embodiments, the MW of x and z are equivalent, and the MW of w is different. In still further embodiments, the MW of x and w are equivalent, and the MW of z is different. In further embodiments, the MW of w and z are equivalent, and the MW of x is different. In still further embodiments, the MW of x, w and z are different. In multi-arm PEG IL-15 molecules, the total size (MW) of the PEG is attributable to the sum of the Mw of the x, w and z components. By way of example, in some embodiments of a multi-arm PEG IL-15 molecule comprising a 50 kDa PEG, x and z can each be 20 kDa and w can be 10 kDa; in other embodiments x and w can each be 20 kDa and z can be 10 kDa; and in further embodiments w and z can each be 20 kDa and x can be 10 kDa. Examples of linkers and PEGs are described herein.

Other embodiments of the present disclosure contemplate multi-functional PEG IL-15 molecules. In such embodiments, two or more IL-15 are covalently attached, optionally via a linker, to a PEG that complexes the two or more IL15. A bifunctional molecule comprises two IL-15 covalently linked to each other through a PEG, a tri-functional molecule comprises three IL-15 covalently linked to each other through PEG, a tetra-functional molecule comprises four IL-15 covalently linked to each other through PEG, and so forth. For purposes of the present disclosure, a multi-functional PEG IL-15 molecule may be represented by the following formulas:

By way of example, for the bifunctional PEG IL-15 molecule, D is a PEG covalently attached to each IL-15 through a PEG of any therapeutically acceptable MW. The PEG may optionally be attached to one or both of the IL-15 through a linker. Examples of linkers and PEGs are described herein.

By way of further example, for the tetra-functional PEG IL-15 molecule, the A₁A₂A₃A₄ complex represents a PEG of any therapeutically acceptable MW covalently attached to each IL-15. The PEG may optionally be attached to one or more of the IL-15 through a linker. Each A₁, A₂, A₃ and A₄ may be of the same or different MW. Thus, for example, for a 40 kDa PEG each A₁, A₂, A₃ and A₄ may be 10 kDa; A₁ and A₂ can both be 15 kDa, and A₃ and A₄ can both be 5 kDa; A₁ can be 2.5 kDa, A₂ can be 7.5 kDa, A₃ can be 10 kDa and A₄ can be 20 kDa₄; and so forth. Examples of linkers and PEGs are described herein.

Pegylation Process Considerations

The primary pegylation processes used for protein conjugation can be broadly classified into two types—a solution phase batch process and an on-column fed-batch process (see Fee, C. J. and Van Alstine, J. M. (2006) Chemical Engineering Science 61(3)924-39). The commonly adopted batch process involves the mixing of reagents together in a suitable buffer solution, preferably at a temperature between 4 and 6 ° C., followed by the separation and purification of the desired product using a suitable technique based on its physicochemical properties, including size exclusion chromatography (SEC), ion exchange chromatography (IEX), hydrophobic interaction chromatography (HIC), membranes or aqueous two-phase systems. The batch process typically entails prolonged contact between reacting species and products that results in multiple conjugations and gives rise to a number of PEG isomers. A heterogeneous product mixture results, constituting unreacted starting materials, hydrolyzed activating agents and a wide range of pegylated products with varying degrees of conjugation. Extensive multistep purifications and downstream processing are often required to isolate the desired product, significantly decreasing overall yields. The high cost of the therapeutic proteins, along with the cost of separating the desired pegylated protein from the reaction mixtures, makes the products extremely expensive, often limiting the application of this approach.

Several on-column pegylation techniques have been utilized with the goal of improving the product profile and specificity of conjugation. For example, a site-specific solid phase peptide pegylation may be used in which the peptide sequence is tethered onto a Rink amide MBHA-resin and is conjugated with a PEG derivative through a side chain lysine or aspartic acid; thereafter, the mono pegylated peptide can be cleaved off from the resin using trifluoroacetic acid (TFA). However, solid-phase synthesis is not practical for large proteins and the harsh chemicals, such as TFA, required for the release of solid-linked pegylated products; as a result, application of this methodology is often not viable with highly sensitive species. Alternatively, ion exchange interactions between protein and ion exchange resins may be used to isolate the pegylated species of interest.

Other on-column pegylation methodologies include size exclusion reaction chromatography (SERC), which incorporates the principle of SEC in separating various molecular sized species based on their different linear velocities through a column packed with porous beads. In this method, activated PEG and protein form a transient in-situ moving reaction zone within the column, in which the pegylated protein, having a larger size than either of the reagents, moves ahead of the reaction zone, thus limiting its residence time in contact with activated PEG and reducing over-pegylation.

PEG Prodrug Conjugates as Drug-Delivery Systems

Two primary approaches are used to target polymeric drugs to a desired location(s): passive targeting and active targeting. These approaches are most commonly used to deliver anticancer drugs to a tumor or cancer cells.

Passive drug targeting. Passive targeting effects drug delivery to the targeted site by conjugating the drug with a polymer, which releases the drug outside the targeted site due to altered environmental conditions. Tumors and many inflamed areas of body have hyperpermeable vasculature and poor lymphatic drainage, which passively provides increased retention of macromolecules into tumors and inflamed body areas. This phenomenon, referred to as enhanced permeability and retention (EPR) effect, is primarily utilized for passive targeting due to accumulation of prodrug into tumors or inflamed areas. Low molecular drugs covalently coupled with high-molecular-weight carriers are inefficiently eliminated due to hampered lymphatic drainage and therefore accumulate in tumors. The EPR effect enhances the passive targeting ability due to higher accumulation rate of drug in tumors, and the prodrug slowly releases drug molecules which provide high bioavailability and low systemic toxicity. [See, e.g., Haag, R. and Kratz, F (2006) Angewandte Chemie—Intl Ed, 45(8):1198-1215].

Active targeting. The active targeting approach is based on interaction between specific biological pairs (e.g., ligand-receptor, antigen-antibody, and enzyme-substrate). It is achieved by attaching targeting agents that bind to specific receptors on the cell surface with the prodrug by a variety of conjugation chemistries. Most widely used targeting moieties are peptide ligands, sugar residues, antibodies, and aptamers specific to particular receptors, selectins, antigens, and mRNAs expressed in targeted cells or organs. The interaction between targeting moieties and their target molecules results in uptake of the drug by either internalization of the prodrug itself, wherein the drug is cleaved intracellularly after endocytosis, or internalization of the drug into targeted cells, wherein the drug is cleaved extracellular by various endocytosis and phagocytosis pathways (see, e.g., Dharap, S. (2003) Journal of Controlled Release 91(1-2):61-73).

Incorporation of linkers into prodrug conjugates. The terms “linker” and “spacer” are used in the polymer technology space and, unless otherwise indicated, for purposes of the present disclosure are used interchangeably. Amino acid spacers such as alanine, glycine, and small peptides are most commonly used due to their chemical versatility for covalent conjugation and biodegradability. Heterobifunctional coupling agents containing succinimidyl have also been used. A detailed description of linkers is set forth elsewhere herein.

In the construction of a prodrug, linkers may be used to fuse the drug with the polymer (e.g., PEG) to decrease the crowding effect, to increase the reactivity, and reduce steric hindrance (Khandare, J. and Minko, T. (2006) Progress in Polymer Science 31(4):359-97). The use of a linker can also enhance ligand-protein binding and provide multiple binding sites. Preferred linkers are stable during conjugate transport and are able to release the bioactive agent at an appropriate site of action.

### Therapeutic and Prophylactic Uses

The present disclosure contemplates the use of the IL-15 polypeptides described herein (e.g., PEG-IL-15) in the treatment or prevention of a broad range of diseases, disorders and/or conditions, and/or the symptoms thereof. While particular uses are described in detail hereafter, it is to be understood that the present disclosure is not so limited. Furthermore, although general categories of particular diseases, disorders and conditions are set forth hereafter, some of the diseases, disorders and conditions may be a member of more than one category, and others may not be a member of any of the disclosed categories.

As discussed in more detail below, IL-15 has been shown to play a role in diseases, disorders and conditions associated with immune and inflammatory function (e.g., autoimmune-related disorders (e.g., rheumatoid arthritis), sarcoidosis, inflammatory bowel disease, and transplant rejection); cancer (e.g., leukemias, lymphoproliferative disorders, and solid tumors); and infectious diseases (e.g., HIV). [See, e.g., Fehniger, et al., Blood 97(1) (Jan. 1, 2001)].

Immune and Inflammatory Conditions. In some embodiments, the present disclosure contemplates suppression of the immune system and treatment of immune-related diseases, disorders and conditions. As used herein, terms such as “immune disease”, “immune condition”, “immune disorder”, “inflammatory disease”, “inflammatory condition”, “inflammatory disorder” and the like are meant to broadly encompass any immune- or inflammatory-related condition (e.g., pathological inflammation and autoimmune diseases). Such conditions frequently are inextricably intertwined with other diseases, disorders and conditions. By way of example, an “immune condition” may refer to proliferative conditions, such as cancer, tumors, and angiogenesis; including infections (acute and chronic), tumors, and cancers that resist eradication by the immune system.

The IL-15 peptides described herein may be used to suppress immune function via the administration of an amount effective to inhibit one or more of the cellular events that normally occurs as a consequence of the interaction between wild-type IL-15 and the IL-15 receptor complex. Alternatively, a nucleic acid molecule encoding the IL-15 peptides described herein or recombinant cells expressing the IL-15 peptides described herein may be administered. In particular embodiments, the IL-15 peptides bind the IL-15 receptor complex with an affinity similar to wild-type IL-15, but fail to activate cell signal transduction. It is advantageous that the IL-15 peptides effectively compete with wild-type IL-15 and inhibit the events normally associated in response to IL-15 signaling.

A non-limiting list of immune- and inflammatory-related diseases, disorders and conditions which may, for example, be caused by inflammatory cytokines, include, arthritis (e.g., rheumatoid arthritis), sarcoidosis, kidney failure, lupus, asthma, psoriasis, colitis, pancreatitis, allergies, surgical complications (e.g., where inflammatory cytokines prevent healing), anemia, and fibromyalgia. Other diseases and disorders which may be associated with chronic inflammation include Alzheimer's disease, congestive heart failure, stroke, aortic valve stenosis, arteriosclerosis, osteoporosis, Parkinson's disease, infections, inflammatory bowel disease (e.g., Crohn's disease and ulcerative colitis), allergic contact dermatitis and other eczemas, systemic sclerosis, transplantation and multiple sclerosis. Some of the aforementioned diseases, disorders and conditions for which an IL-15 molecule may be particularly efficacious (due to, for example, limitations of current therapies) are described in more detail hereafter.

The IL-15 polypeptides of the present disclosure may be particularly effective in the treatment and prevention of inflammatory bowel diseases (IBD). IBD comprises Crohn's disease (CD) and ulcerative colitis (UC), both of which are idiopathic chronic diseases that can affect any part of the gastrointestinal tract, and are associated with many untoward effects, and patients with prolonged UC are at an increased risk of developing colon cancer. Current IBD treatments are aimed at controlling inflammatory symptoms, and while certain agents (e.g., corticosteroids, aminosalicylates and standard immunosuppressive agents (e.g., cyclosporine, azathioprine, and methotrexate)) have met with limited success, long-term therapy may cause liver damage (e.g., fibrosis or cirrhosis) and bone marrow suppression, and patients often become refractory to such treatments.

Psoriasis, a constellation of common immune-mediated chronic skin diseases, affects more than 4.5 million people in the U.S., of which 1.5 million are considered to have a moderate-to severe form of the disease. Furthermore, over 10% of patients with psoriasis develop psoriatic arthritis, which damages the bone and connective tissue around the joints. An improved understanding of the underlying physiology of psoriasis has resulted in the introduction of agents that, for example, target the activity of T lymphocytes and cytokines responsible for the inflammatory nature of the disease. Such agents include the TNF-α inhibitors (also used in the treatment of rheumatoid arthritis (RA)), including ENBREL (etanercept), REMICADE (infliximab) and HUMIRA (adalimumab)), and T-cell inhibitors such as AMEVIVE (alefacept) and RAPTIVA (efalizumab). Though several of these agents are effective to some extent in certain patient populations, none have been shown to effectively treat all patients.

Rheumatoid Arthritis (RA), which is generally characterized by chronic inflammation in the membrane lining (the synovium) of the joints, affects approximately 1% of the U.S. population (˜2.1 million people). Further understanding of the role of cytokines, including TNF-α and IL-1, in the inflammatory process has enabled the development and introduction of a new class of disease-modifying antirheumatic drugs (DMARDs). Agents (some of which overlap with treatment modalities for other indications) include ENBREL (etanercept), REMICADE (infliximab), HUMIRA (adalimumab) and KINERET (anakinra). Though some of these agents relieve symptoms, inhibit progression of structural damage, and improve physical function in particular patient populations, there is still a need for alternative agents with improved efficacy, complementary mechanisms of action, and fewer/less severe adverse effects.

Transplant rejection of organs and tissues has been found to involve an IL-15—related component in certain situations. Rejection is an adaptive immune response that is mediated by both cellular immunity and humoral immunity, along with components of innate immune response. Different types of transplanted organs and tissues often have different balances of rejection mechanisms. Kidney, heart, bone marrow, skin, and blood are the organs and tissues most frequently involved in transplant rejection. Treatment of transplant rejections is often dictated by the medical category of rejection (e.g., hyperacute, acute, or chronic).

Immunosuppressive therapy constitutes the primary means of treating transplant rejection. Therapy is generally initiated with corticosteroids (e.g., prednisone). Combination therapy typically entails the addition of a calcineurin inhibitor (e.g., cyclosporin and tacrolimus) and an anti-proliferative agent (e.g., azathioprine). Antibodies specific to particular immune components may be added to immunosuppressive therapy; antibody therapeutics include monoclonal anti-IL-2Rα receptor antibodies (e.g., daclizumad) and monoclonal anti-CD20 antibodies (e.g., rituximab). Though helpful in many situations, alternative treatment modalities such as IL-15 related agents are needed.

Subjects suffering from multiple sclerosis (MS), a seriously debilitating autoimmune disease comprising multiple areas of inflammation and scarring of the myelin in the brain and spinal cord, may be particularly helped by the IL-15 polypeptides described herein, as current treatments only alleviate symptoms or delay the progression of disability.

Elevated serum levels of IL-15 have been observed during hepatitis C-induced liver diseases, and in liver cirrhosis and chronic hepatitis. IL-15 levels are particularly elevated in subjects suffering from hepatocellular carcinoma.

Similarly, the IL-15 polypeptides may be particularly advantageous for subjects afflicted with neurodegenerative disorders, such as Alzheimer's disease (AD), a brain disorder that seriously impairs patients' thought, memory, and language processes; Parkinson's disease (PD), a progressive disorder of the CNS characterized by, for example, abnormal movement, rigidity and tremor; and diabetes mellitus. These disorders are progressive and debilitating, and no curative agents are available.

Cancer and Related Conditions. In accordance with the present disclosure, an IL-15 molecule (e.g., peptide) described herein can be used to treat a subject having undesirable proliferation of cells that express an IL-15 receptor. Alternatively, a nucleic acid molecule encoding the IL-15 peptides described herein or recombinant cells expressing the IL-15 peptides described herein may be administered. Though an understanding of the underlying mechanism of action by which IL-15 exerts an anti-proliferative effect is not required to practice the present disclosure, cellular proliferation may be inhibited by complement-directed cytolysis or antibody-dependent cellular toxicity.

The IL-15 peptides described herein can be used to treat or prevent a proliferative condition or disorder, including a cancer, for example, cancer of the uterus, cervix, breast, prostate, testes, gastrointestinal tract (e.g., esophagus, oropharynx, stomach, small or large intestines, colon, or rectum), kidney, renal cell, bladder, bone, bone marrow, skin, head or neck, liver, gall bladder, heart, lung, pancreas, salivary gland, adrenal gland, thyroid, brain (e.g., gliomas), ganglia, central nervous system (CNS) and peripheral nervous system (PNS), and cancers of the hematopoietic system and the immune system (e.g., spleen or thymus). The present disclosure also provides methods of treating or preventing other cancer-related diseases, disorders or conditions, including, for example, immunogenic tumors, non-immunogenic tumors, dormant tumors, virus-induced cancers (e.g., epithelial cell cancers, endothelial cell cancers, squamous cell carcinomas and papillomavirus), adenocarcinomas, lymphomas (e.g., cutaneous T-cell lymphoma (CTCL), carcinomas, melanomas, leukemias, myelomas, sarcomas, teratocarcinomas, chemically-induced cancers, metastasis, and angiogenesis.

In particular embodiments, the tumor or cancer is colon cancer, ovarian cancer, breast cancer, melanoma, lung cancer, glioblastoma, or leukemia (e.g., HTLV-1—mediated adult T-cell leukemia). The use of the term(s) cancer-related diseases, disorders and conditions is meant to refer broadly to conditions that are associated, directly or indirectly, with cancer, and includes, e.g., angiogenesis and precancerous conditions such as dysplasia.

In some embodiments, the present disclosure provides methods for treating a proliferative condition, cancer, tumor, or precancerous condition with an IL-15 molecule and at least one additional therapeutic or diagnostic agent, examples of which are set forth elsewhere herein.

Viral and Bacterial Conditions. There has been increased interest in the role of IL-15 in viral and bacterial diseases, disorders and conditions. IL-15 has been postulated to produce both stimulatory and inhibitory effects depending on its receptor binding activity and other factors.

Regarding human immunodeficiency virus (HIV), IL-15, through its ability to mimic the actions of IL-2, has two conflicting effects. One effect is the potentially beneficial enhancement of immune function, while the other effect is the potentially detrimental activation of HIV replication. These opposing effects are also present in other viral-related disorders. A close temporal correlation was observed between IL-15 levels and fluctuations in viral load.

The present disclosure contemplates the use of the IL-15 polypeptides in the treatment and/or prevention of any viral disease, disorder or condition for which treatment with IL-15 may be beneficial. Examples of viral diseases, disorders and conditions that are contemplated include Epstein-Barr virus, hepatitis B, hepatitis C, HIV, herpes simplex virus and cytomegalovirus (CMV).

IL-15 has recently been associated with certain bacterial and other invasive infections. By way of example, reports indicate that administration of recombinant IL-15 before infection caused by, e.g., Salmonella and Plasmodium falciparum improves host defense against, and clearance of, the organism.

### Pharmaceutical Compositions

The IL-15 polypeptides of the present disclosure may be in the form of compositions suitable for administration to a subject. In general, such compositions are “pharmaceutical compositions” comprising IL-15 and one or more pharmaceutically acceptable or physiologically acceptable diluents, carriers or excipients. In certain embodiments, the IL-15 polypeptides are present in a therapeutically acceptable amount. The pharmaceutical compositions may be used in the methods of the present disclosure; thus, for example, the pharmaceutical compositions can be administered ex vivo or in vivo to a subject in order to practice the therapeutic and prophylactic methods and uses described herein.

The pharmaceutical compositions of the present disclosure can be formulated to be compatible with the intended method or route of administration; exemplary routes of administration are set forth herein. Furthermore, the pharmaceutical compositions may be used in combination with other therapeutically active agents or compounds as described herein in order to treat or prevent the diseases, disorders and conditions as contemplated by the present disclosure.

The pharmaceutical compositions typically comprise a therapeutically effective amount of an IL-15 polypeptide contemplated by the present disclosure and one or more pharmaceutically and physiologically acceptable formulation agents. Suitable pharmaceutically acceptable or physiologically acceptable diluents, carriers or excipients include, but are not limited to, antioxidants (e.g., ascorbic acid and sodium bisulfate), preservatives (e.g., benzyl alcohol, methyl parabens, ethyl or n-propyl, p-hydroxybenzoate), emulsifying agents, suspending agents, dispersing agents, solvents, fillers, bulking agents, detergents, buffers, vehicles, diluents, and/or adjuvants. For example, a suitable vehicle may be physiological saline solution or citrate buffered saline, possibly supplemented with other materials common in pharmaceutical compositions for parenteral administration. Neutral buffered saline or saline mixed with serum albumin are further exemplary vehicles. Those skilled in the art will readily recognize a variety of buffers that can be used in the pharmaceutical compositions and dosage forms contemplated herein. Typical buffers include, but are not limited to, pharmaceutically acceptable weak acids, weak bases, or mixtures thereof. As an example, the buffer components can be water soluble materials such as phosphoric acid, tartaric acids, lactic acid, succinic acid, citric acid, acetic acid, ascorbic acid, aspartic acid, glutamic acid, and salts thereof. Acceptable buffering agents include, for example, a Tris buffer, N-(2-Hydroxyethyl)piperazine-N′-(2-ethanesulfonic acid) (HEPES), 2-(N-Morpholino)ethanesulfonic acid (MES), 2-(N-Morpholino) ethanesulfonic acid sodium salt (MES), 3-(N-Morpholino)propanesulfonic acid (MOPS), and N-tris[Hydroxymethyl]methyl-3-aminopropanesulfonic acid (TAPS).

After a pharmaceutical composition has been formulated, it may be stored in sterile vials as a solution, suspension, gel, emulsion, solid, or dehydrated or lyophilized powder. Such formulations may be stored either in a ready-to-use form, a lyophilized form requiring reconstitution prior to use, a liquid form requiring dilution prior to use, or other acceptable form. In some embodiments, the pharmaceutical composition is provided in a single-use container (e.g., a single-use vial, ampoule, syringe, or autoinjector (similar to, e.g., an EpiPen®)), whereas a multi-use container (e.g., a multi-use vial) is provided in other embodiments. Any drug delivery apparatus may be used to deliver IL-15, including implants (e.g., implantable pumps) and catheter systems, slow injection pumps and devices, all of which are well known to the skilled artisan. Depot injections, which are generally administered subcutaneously or intramuscularly, may also be utilized to release the polypeptides disclosed herein over a defined period of time. Depot injections are usually either solid- or oil-based and generally comprise at least one of the formulation components set forth herein. One of ordinary skill in the art is familiar with possible formulations and uses of depot injections.

The pharmaceutical compositions may be in the form of a sterile injectable aqueous or oleagenous suspension. This suspension may be formulated according to the known art using those suitable dispersing or wetting agents and suspending agents mentioned herein. The sterile injectable preparation may also be a sterile injectable solution or suspension in a non-toxic parenterally-acceptable diluent or solvent, for example, as a solution in 1,3-butane diol. Acceptable diluents, solvents and dispersion media that may be employed include water, Ringer's solution, isotonic sodium chloride solution, Cremophor ELTM (BASF, Parsippany, NJ) or phosphate buffered saline (PBS), ethanol, polyol (e.g., glycerol, propylene glycol, and liquid polyethylene glycol), and suitable mixtures thereof. In addition, sterile, fixed oils are conventionally employed as a solvent or suspending medium. For this purpose, any bland fixed oil may be employed, including synthetic mono- or diglycerides. Moreover, fatty acids such as oleic acid, find use in the preparation of injectables. Prolonged absorption of particular injectable formulations can be achieved by including an agent that delays absorption (e.g., aluminum monostearate or gelatin).

The pharmaceutical compositions containing the active ingredient may be in a form suitable for oral use, for example, as tablets, capsules, troches, lozenges, aqueous or oily suspensions, dispersible powders or granules, emulsions, hard or soft capsules, or syrups, solutions, microbeads or elixirs. Pharmaceutical compositions intended for oral use may be prepared according to any method known in the art for the manufacture of pharmaceutical compositions, and such compositions may contain one or more agents such as, for example, sweetening agents, flavoring agents, coloring agents and preserving agents in order to provide pharmaceutically elegant and palatable preparations. Tablets, capsules and the like contain the active ingredient in admixture with non-toxic pharmaceutically acceptable excipients which are suitable for the manufacture of tablets. These excipients may be, for example, diluents, such as calcium carbonate, sodium carbonate, lactose, calcium phosphate or sodium phosphate; granulating and disintegrating agents, for example, corn starch, or alginic acid; binding agents, for example starch, gelatin or acacia; and lubricating agents, for example magnesium stearate, stearic acid or talc.

The tablets, capsules and the like suitable for oral administration may be uncoated or coated by known techniques to delay disintegration and absorption in the gastrointestinal tract and thereby provide a sustained action. For example, a time-delay material such as glyceryl monostearate or glyceryl distearate may be employed. They may also be coated by techniques known in the art to form osmotic therapeutic tablets for controlled release. Additional agents include biodegradable or biocompatible particles or a polymeric substance such as polyesters, polyamine acids, hydrogel, polyvinyl pyrrolidone, polyanhydrides, polyglycolic acid, ethylene-vinyl acetate, methylcellulose, carboxymethylcellulose, protamine sulfate, or lactide/glycolide copolymers, polylactide/glycolide copolymers, or ethylenevinylacetate copolymers in order to control delivery of an administered composition. For example, the oral agent can be entrapped in microcapsules prepared by coacervation techniques or by interfacial polymerization, by the use of hydroxymethylcellulose or gelatin-microcapsules or poly (methylmethacrolate) microcapsules, respectively, or in a colloid drug delivery system. Colloidal dispersion systems include macromolecule complexes, nano-capsules, microspheres, microbeads, and lipid-based systems, including oil-in-water emulsions, micelles, mixed micelles, and liposomes. Methods for the preparation of the above-mentioned formulations will be apparent to those skilled in the art.

Formulations for oral use may also be presented as hard gelatin capsules wherein the active ingredient is mixed with an inert solid diluent, for example, calcium carbonate, calcium phosphate, kaolin or microcrystalline cellulose, or as soft gelatin capsules wherein the active ingredient is mixed with water or an oil medium, for example peanut oil, liquid paraffin, or olive oil.

Aqueous suspensions contain the active materials in admixture with excipients suitable for the manufacture thereof. Such excipients can be suspending agents, for example sodium carboxymethylcellulose, methylcellulose, hydroxy-propylmethylcellulose, sodium alginate, polyvinyl-pyrrolidone, gum tragacanth and gum acacia; dispersing or wetting agents, for example a naturally-occurring phosphatide (e.g., lecithin), or condensation products of an alkylene oxide with fatty acids (e.g., polyoxy-ethylene stearate), or condensation products of ethylene oxide with long chain aliphatic alcohols (e.g., for heptadecaethyleneoxycetanol), or condensation products of ethylene oxide with partial esters derived from fatty acids and a hexitol (e.g., polyoxyethylene sorbitol monooleate), or condensation products of ethylene oxide with partial esters derived from fatty acids and hexitol anhydrides (e.g., polyethylene sorbitan monooleate). The aqueous suspensions may also contain one or more preservatives.

Oily suspensions may be formulated by suspending the active ingredient in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified herein.

The pharmaceutical compositions of the present disclosure may also be in the form of oil-in-water emulsions. The oily phase may be a vegetable oil, for example olive oil or arachis oil, or a mineral oil, for example, liquid paraffin, or mixtures of these. Suitable emulsifying agents may be naturally occurring gums, for example, gum acacia or gum tragacanth; naturally occurring phosphatides, for example, soy bean, lecithin, and esters or partial esters derived from fatty acids; hexitol anhydrides, for example, sorbitan monooleate; and condensation products of partial esters with ethylene oxide, for example, polyoxyethylene sorbitan monooleate.

Formulations can also include carriers to protect the composition against rapid degradation or elimination from the body, such as a controlled release formulation, including implants, liposomes, hydrogels, prodrugs and microencapsulated delivery systems. For example, a time delay material such as glyceryl monostearate or glyceryl stearate alone, or in combination with a wax, may be employed.

The present disclosure contemplates the administration of the IL-15 polypeptides in the form of suppositories for rectal administration. The suppositories can be prepared by mixing the drug with a suitable non-irritating excipient which is solid at ordinary temperatures but liquid at the rectal temperature and will therefore melt in the rectum to release the drug. Such materials include, but are not limited to, cocoa butter and polyethylene glycols.

The IL-15 polypeptides contemplated by the present disclosure may be in the form of any other suitable pharmaceutical composition (e.g., sprays for nasal or inhalation use) currently known or developed in the future.

The concentration of a polypeptide or fragment thereof in a formulation can vary widely (e.g., from less than about 0.1%, usually at or at least about 2% to as much as 20% to 50% or more by weight) and will usually be selected primarily based on fluid volumes, viscosities, and subject-based factors in accordance with, for example, the particular mode of administration selected.

### Routes of Administration

The present disclosure contemplates the administration of IL-15 molecules, and compositions thereof, in any appropriate manner. Suitable routes of administration include parenteral (e.g., intramuscular, intravenous, subcutaneous (e.g., injection or implant), intraperitoneal, intracisternal, intraarticular, intraperitoneal, intracerebral (intraparenchymal) and intracerebroventricular), oral, nasal, vaginal, sublingual, intraocular, rectal, topical (e.g., transdermal), sublingual and inhalation. Depot injections, which are generally administered subcutaneously or intramuscularly, may also be utilized to release the IL-15 molecules disclosed herein over a defined period of time.

Particular embodiments of the present disclosure contemplate parenteral administration, and in further particular embodiments the parenteral administration is subcutaneous.

### Combination Therapy

The present disclosure contemplates the use of IL-15 molecules in combination with one or more active therapeutic agents (e.g., cytokines) or other prophylactic or therapeutic modalities (e.g., radiation). In such combination therapy, the various active agents frequently have different, complementary mechanisms of action. Such combination therapy may be especially advantageous by allowing a dose reduction of one or more of the agents, thereby reducing or eliminating the adverse effects associated with one or more of the agents. Furthermore, such combination therapy may have a synergistic therapeutic or prophylactic effect on the underlying disease, disorder, or condition.

As used herein, “combination” is meant to include therapies that can be administered separately, for example, formulated separately for separate administration (e.g., as may be provided in a kit), and therapies that can be administered together in a single formulation (i.e., a “co-formulation”).

In certain embodiments, the IL-15 polypeptides and the one or more active therapeutic agents or other prophylactic or therapeutic modalities are administered or applied sequentially, e.g., where one agent is administered prior to one or more other agents. In other embodiments, the IL-15 polypeptides and the one or more active therapeutic agents or other prophylactic or therapeutic modalities are administered simultaneously, e.g., where two or more agents are administered at or about the same time; the two or more agents may be present in two or more separate formulations or combined into a single formulation (i.e., a co-formulation). Regardless of whether the two or more agents are administered sequentially or simultaneously, they are considered to be administered in combination for purposes of the present disclosure.

The IL-15 polypeptides of the present disclosure may be used in combination with at least one other (active) agent in any manner appropriate under the circumstances. In one embodiment, treatment with the at least one active agent and at least one IL-15 polypeptide of the present disclosure is maintained over a period of time. In another embodiment, treatment with the at least one active agent is reduced or discontinued (e.g., when the subject is stable), while treatment with the IL-15 polypeptide of the present disclosure is maintained at a constant dosing regimen. In a further embodiment, treatment with the at least one active agent is reduced or discontinued (e.g., when the subject is stable), while treatment with the IL-15 polypeptide of the present disclosure is reduced (e.g., lower dose, less frequent dosing or shorter treatment regimen). In yet another embodiment, treatment with the at least one active agent is reduced or discontinued (e.g., when the subject is stable), and treatment with the IL-15 polypeptide of the present disclosure is increased (e.g., higher dose, more frequent dosing or longer treatment regimen). In yet another embodiment, treatment with the at least one active agent is maintained and treatment with the IL-15 polypeptide of the present disclosure is reduced or discontinued (e.g., lower dose, less frequent dosing or shorter treatment regimen). In yet another embodiment, treatment with the at least one active agent and treatment with the IL-15 polypeptide of the present disclosure are reduced or discontinued (e.g., lower dose, less frequent dosing or shorter treatment regimen).

Immune and Inflammatory Conditions. The present disclosure provides methods for treating and/or preventing immune- and/or inflammatory-related diseases, disorders and conditions, as well as disorders associated therewith, with an IL-15 molecule and at least one additional therapeutic or diagnostic agent.

Examples of therapeutic agents useful in combination therapy include, but are not limited to, the following: non-steroidal anti-inflammatory drug (NSAID) such as aspirin, ibuprofen, and other propionic acid derivatives (alminoprofen, benoxaprofen, bucloxic acid, carprofen, fenbufen, fenoprofen, fluprofen, flurbiprofen, indoprofen, ketoprofen, miroprofen, naproxen, oxaprozin, pirprofen, pranoprofen, suprofen, tiaprofenic acid, and tioxaprofen), acetic acid derivatives (indomethacin, acemetacin, alclofenac, clidanac, diclofenac, fenclofenac, fenclozic acid, fentiazac, fuirofenac, ibufenac, isoxepac, oxpinac, sulindac, tiopinac, tolmetin, zidometacin, and zomepirac), fenamic acid derivatives (flufenamic acid, meclofenamic acid, mefenamic acid, niflumic acid and tolfenamic acid), biphenylcarboxylic acid derivatives (diflunisal and flufenisal), oxicams (isoxicam, piroxicam, sudoxicam and tenoxican), salicylates (acetyl salicylic acid, sulfasalazine) and the pyrazolones (apazone, bezpiperylon, feprazone, mofebutazone, oxyphenbutazone, phenylbutazone). Other combinations include cyclooxygenase-2 (COX-2) inhibitors.

Other active agents for combination include steroids such as prednisolone, prednisone, methylprednisolone, betamethasone, dexamethasone, or hydrocortisone. Such a combination may be especially advantageous since one or more adverse effects of the steroid can be reduced or even eliminated by tapering the steroid dose required.

Additional examples of active agents that may be used in combinations for treating, for example, rheumatoid arthritis, include cytokine suppressive anti-inflammatory drug(s) (CSAIDs); antibodies to, or antagonists of, other human cytokines or growth factors, for example, TNF, LT, IL-10, IL-2, IL-6, IL-7, IL-8, IL-10, IL-16, IL-18, EMAP-II, GM-CSF, FGF, or PDGF.

Particular combinations of active agents may interfere at different points in the autoimmune and subsequent inflammatory cascade, and include TNF antagonists such as chimeric, humanized or human TNF antibodies, REMICADE, anti-TNF antibody fragments (e.g., CDP870), and soluble p55 or p75 TNF receptors, derivatives thereof, p75TNFRIgG (ENBREL.) or p55TNFR1gG (LENERCEPT), soluble IL-13 receptor (sIL-13), and also TNFα-converting enzyme (TACE) inhibitors; similarly, IL-1 inhibitors (e.g., Interleukin-1-converting enzyme inhibitors) may be effective. Other combinations include Interleukin 11, anti-P7s and p-selectin glycoprotein ligand (PSGL). Other examples of agents useful in combination with the IL-15 polypeptides described herein include interferon-(31α (AVONEX); interferon-β1b (BETASERON); copaxone; hyperbaric oxygen; intravenous immunoglobulin; clabribine; and antibodies to, or antagonists of, other human cytokines or growth factors (e.g., antibodies to CD40 ligand and CD80).

The present disclosure encompasses pharmaceutically acceptable salts, acids or derivatives of any of the above.

Cancer and Related Conditions. The present disclosure provides methods for treating and/or preventing a proliferative condition; a cancer, tumor, or precancerous disease, disorder or condition with an IL-15 molecule and at least one additional therapeutic or diagnostic agent.

Examples of chemotherapeutic agents include, but are not limited to, alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamime; nitrogen mustards such as chiorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, caminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenishers such as frolinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; razoxane; sizofiran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (Ara-C); cyclophosphamide; thiotepa; taxoids, e.g., paclitaxel and doxetaxel; chlorambucil; gemcitabine; 6-thioguanine; mercaptopurine; methotrexate; platinum and platinum coordination complexes such as cisplatin and carboplatin; vinblastine; etoposide (VP-16); ifosfamide; mitomycin C; mitoxantrone; vincristine; vinorelbine; navelbine; novantrone; teniposide; daunomycin; aminopterin; xeloda; ibandronate; CPT11; topoisomerase inhibitors; difluoromethylornithine (DMFO); retinoic acid; esperamicins; capecitabine; and pharmaceutically acceptable salts, acids or derivatives of any of the above.

Chemotherapeutic agents also include anti-hormonal agents that act to regulate or inhibit hormonal action on tumors such as anti-estrogens, including for example tamoxifen, raloxifene, aromatase inhibiting 4(5)-imidazoles, 4-hydroxytamoxifen, trioxifene, keoxifene, onapristone, and toremifene; and antiandrogens such as flutamide, nilutamide, bicalutamide, leuprolide, and goserelin; and pharmaceutically acceptable salts, acids or derivatives of any of the above. In certain embodiments, combination therapy comprises administration of a hormone or related hormonal agent.

Additional treatment modalities that may be used in combination with the IL-15 polypeptides include a cytokine or cytokine antagonist, such as IL-12, INFa, or anti-epidermal growth factor receptor, radiotherapy, a monoclonal antibody against another tumor antigen, a complex of a monoclonal antibody and toxin, a T-cell adjuvant, bone marrow transplant, or antigen presenting cells (e.g., dendritic cell therapy). Vaccines (e.g., as a soluble protein or as a nucleic acid encoding the protein) are also provided herein.

The present disclosure encompasses pharmaceutically acceptable salts, acids or derivatives of any of the above.

Viral and Bacterial Conditions. The present disclosure provides methods for treating and/or preventing viral diseases, disorders and conditions, as well as disorders associated therewith, with an IL-15 molecule and at least one additional therapeutic or diagnostic agent (e.g., one or more other antiviral agents and/or one or more agents not associated with viral therapy).

Such combination therapy includes anti-viral agents targeting various viral life-cycle stages and having different mechanisms of action, including, but not limiting to, the following: inhibitors of viral uncoating (e.g., amantadine and rimantidine); reverse transcriptase inhibitors (e.g., acyclovir, zidovudine, and lamivudine); agents that target integrase; agents that block attachment of transcription factors to viral DNA; agents (e.g., antisense molecules) that impact translation (e.g., fomivirsen); agents that modulate translation/ribozyme function; protease inhibitors; viral assembly modulators (e.g., rifampicin); and agents that prevent release of viral particles (e.g., zanamivir and oseltamivir). Treatment and/or prevention of certain viral infections (e.g., HIV) frequently entail a group (“cocktail”) of antiviral agents.

Other antiviral agents contemplated for use in combination with IL-15 polypeptides include, but are not limited to, the following: abacavir, adefovir, amantadine, amprenavir, ampligen, arbidol, atazanavir, atripla, boceprevirertet, cidofovir, combivir, darunavir, delavirdine, didanosine, docosanol, edoxudine, efavirenz, emtricitabine, enfuvirtide, entecavir, famciclovir, fosamprenavir, foscarnet, fosfonet, ganciclovir, ibacitabine, imunovir, idoxuridine, imiquimod, indinavir, inosine, various interferons (e.g., peginterferon alfa-2a), lopinavir, loviride, maraviroc, moroxydine, methisazone, nelfinavir, nevirapine, nexavir, penciclovir, peramivir, pleconaril, podophyllotoxin, raltegravir, ribavirin, ritonavir, pyramidine, saquinavir, stavudine, telaprevir, tenofovir, tipranavir, trifluridine, trizivir, tromantadine, truvada, valaciclovir, valganciclovir, vicriviroc, vidarabine, viramidine, and zalcitabine.

IL-15 treatment of the Salmenella genus of rod-shaped Gram-negative bacteria is thought to be most effective in combination with vaccines currently under development. In regards to combination therapy for the treatment of Plasmodium falciparum parasite, the antimalarial medications (e.g., cholorquines) ant the artemisininis may be effective in combination therapy with IL-15 peptides.

The present disclosure encompasses pharmaceutically acceptable salts, acids or derivatives of any of the above.

### Dosing

The IL-15 polypeptides of the present disclosure may be administered to a subject in an amount that is dependent upon, for example, the goal of administration (e.g., the degree of resolution desired); the age, weight, sex, and health and physical condition of the subject to which the formulation is being administered; the route of administration; and the nature of the disease, disorder, condition or symptom thereof. The dosing regimen may take into consideration the existence, nature, and extent of any adverse effects associated with the agent(s) being administered. Effective dosage amounts and dosage regimens can readily be determined from, for example, safety and dose-escalation trials, in vivo studies (e.g., animal models), and other methods known to the skilled artisan.

In general, dosing parameters dictate that the dosage amount be less than an amount that could be irreversibly toxic to the subject (the maximum tolerated dose (MTD)) and not less than an amount required to produce a measurable effect on the subject. Such amounts are determined by, for example, the pharmacokinetic and pharmacodynamic parameters associated with ADME, taking into consideration the route of administration and other factors.

An effective dose (ED) is the dose or amount of an agent that produces a therapeutic response or desired effect in some fraction of the subjects taking it. The “median effective dose” or ED50 of an agent is the dose or amount of an agent that produces a therapeutic response or desired effect in 50% of the population to which it is administered. Although the ED50 is commonly used as a measure of reasonable expectance of an agent's effect, it is not necessarily the dose that a clinician might deem appropriate taking into consideration all relevant factors. Thus, in some situations the effective amount is more than the calculated ED50, in other situations the effective amount is less than the calculated ED50, and in still other situations the effective amount is the same as the calculated ED50.

In addition, an effective dose of the IL-15 molecules of the present disclosure may be an amount that, when administered in one or more doses to a subject, produces a desired result relative to a healthy subject. For example, for a subject experiencing a particular disorder, an effective dose may be one that improves a diagnostic parameter, measure, marker and the like of that disorder by at least about 5%, at least about 10%, at least about 20%, at least about 25%, at least about 30%, at least about 40%, at least about 50%, at least about 60%, at least about 70%, at least about 80%, at least about 90%, or more than 90%, where 100% is defined as the diagnostic parameter, measure, marker and the like exhibited by a normal subject. The amount of an IL-15 molecule necessary to treat a disease, disorder or condition described herein is based on the IL-15 activity of the conjugated protein, which can be determined by IL-15 activity assays known in the art.

The therapeutically effective amount of an IL-15 molecule can range from about 0.01 to about 100 μg protein/kg of body weight/day, from about 0.1 to 20 μg protein/kg of body weight/day, from about 0.5 to 10 μg protein/kg of body weight/day, or from about 1 to 4 μg protein/kg of body weight/day. In some embodiments, the therapeutically effective amount of an IL-15 molecule can range from about 1 to 16 μg protein/kg of body weight/day. The present disclosure contemplates the administration of an IL-15 molecule by continuous infusion to delivery, e.g., about 50 to 800 μg protein/kg of body weight/day. The infusion rate may be varied based on evaluation of, for example, adverse effects and blood cell counts.

For administration of an oral agent, the compositions can be provided in the form of tablets, capsules and the like containing from 1.0 to 1000 milligrams of the active ingredient, particularly 1.0, 3.0, 5.0, 10.0, 15.0, 20.0, 25.0, 50.0, 75.0, 100.0, 150.0, 200.0, 250.0, 300.0, 400.0, 500.0, 600.0, 750.0, 800.0, 900.0, or 1000.0 milligrams of the active ingredient.

In certain embodiments, the dosage of the disclosed IL-15 polypeptide is contained in a “unit dosage form”. The phrase “unit dosage form” refers to physically discrete units, each unit containing a predetermined amount of a IL-15 polypeptide of the present disclosure, either alone or in combination with one or more additional agents, sufficient to produce the desired effect. It will be appreciated that the parameters of a unit dosage form will depend on the particular agent and the effect to be achieved.

### Kits

The present disclosure also contemplates kits comprising IL-15, and pharmaceutical compositions thereof. The kits are generally in the form of a physical structure housing various components, as described below, and may be utilized, for example, in practicing the methods described herein.

A kit can include one or more of the IL-15 polypeptides disclosed herein (provided in, e.g., a sterile container), which may be in the form of a pharmaceutical composition suitable for administration to a subject. The IL-15 polypeptides can be provided in a form that is ready for use or in a form requiring, for example, reconstitution or dilution prior to administration. When the IL-15 polypeptides are in a form that needs to be reconstituted by a user, the kit may also include buffers, pharmaceutically acceptable excipients, and the like, packaged with or separately from the IL-15 polypeptides. When combination therapy is contemplated, the kit may contain the several agents separately or they may already be combined in the kit. Each component of the kit may be enclosed within an individual container, and all of the various containers may be within a single package. A kit of the present disclosure may be designed for conditions necessary to properly maintain the components housed therein (e.g., refrigeration or freezing).

A kit may contain a label or packaging insert including identifying information for the components therein and instructions for their use (e.g., dosing parameters, clinical pharmacology of the active ingredient(s), including mechanism of action, pharmacokinetics and pharmacodynamics, adverse effects, contraindications, etc.). Labels or inserts can include manufacturer information such as lot numbers and expiration dates. The label or packaging insert may be, e.g., integrated into the physical structure housing the components, contained separately within the physical structure, or affixed to a component of the kit (e.g., an ampule, tube or vial).

Labels or inserts can additionally include, or be incorporated into, a computer readable medium, such as a disk (e.g., hard disk, card, memory disk), optical disk such as CD- or DVD-ROM/RAM, DVD, MP3, magnetic tape, or an electrical storage media such as RAM and ROM or hybrids of these such as magnetic/optical storage media, FLASH media or memory-type cards. In some embodiments, the actual instructions are not present in the kit, but means for obtaining the instructions from a remote source, e.g., via the internet, are provided.

### Experimental

The following examples are put forth so as to provide those of ordinary skill in the art with a complete disclosure and description of how to make and use the present invention, and are not intended to limit the scope of what the inventors regard as their invention nor are they intended to represent that the experiments below were performed and are all of the experiments that may be performed. It is to be understood that exemplary descriptions written in the present tense were not necessarily performed, but rather that the descriptions can be performed to generate the data and the like described therein. Efforts have been made to ensure accuracy with respect to numbers used (e.g., amounts, temperature, etc.), but some experimental errors and deviations should be accounted for.

Unless indicated otherwise, parts are parts by weight, molecular weight is weight average molecular weight, temperature is in degrees Celsius (° C.), and pressure is at or near atmospheric.

Standard abbreviations are used, including the following: bp=base pair(s); kb=kilobase(s); pl=picoliter(s); s or sec=second(s); min=minute(s); h or hr=hour(s); aa=amino acid(s); kb=kilobase(s); nt=nucleotide(s); ng=nanogram; μg=microgram; mg=milligram; g=gram; kg=kilogram; dl or dL=deciliter; μL or μL=microliter; ml or mL=milliliter; l or L =liter; nM=nanomolar; μM=micromolar; mM=millimolar; M=molar; kDa=kilodalton; i.m.=intramuscular(ly); i.p.=intraperitoneal(ly); s.c.=subcutaneous(ly); QD=daily; BID=twice daily; QW=weekly; QM=monthly; HPLC=high performance liquid chromatography; BW=body weight; U=unit; ns=not statistically significant; PBS=phosphate-buffered saline; PCR=polymerase chain reaction; NHS=N-Hydroxysuccinimide; DMEM=Dulbeco's Modification of Eagle's Medium; GC=genome copy; ELISA=enzyme-linked immuno sorbent assay; EDTA=ethylenediaminetetraacetic acid; PMA=phorbol myristate acetate; rhlL-1532 recombinant human IL-15; LPS=lipopolysaccharide.

### Materials and Methods

The following general materials and methods may be used in the Examples below:

Standard methods in molecular biology are described (see, e.g., Sambrook and Russell (2001) Molecular Cloning, 3^{rd} ed., Cold Spring Harbor Laboratory Press, Cold Spring Harbor, N.Y.; and Ausubel, et al. (2001) Current Protocols in Molecular Biology, Vols. 1-4, John Wiley and Sons, Inc. New York, N.Y., which describes cloning in bacterial cells and DNA mutagenesis (Vol. 1), cloning in mammalian cells and yeast (Vol. 2), glycoconjugates and protein expression (Vol. 3), and bioinformatics (Vol. 4)).

The scientific literature describes methods for protein purification, including immunoprecipitation, chromatography, electrophoresis, centrifugation, and crystallization, as well as chemical analysis, chemical modification, post-translational modification, production of fusion proteins, and glycosylation of proteins (see, e.g., Coligan, et al. (2000) Current Protocols in Protein Science, Vols. 1-2, John Wiley and Sons, Inc., N.Y.).

Production, purification, and fragmentation of polyclonal and monoclonal antibodies are described (e.g., Harlow and Lane (1999) Using Antibodies, Cold Spring Harbor Laboratory Press, Cold Spring Harbor, N.Y.); standard techniques for characterizing ligand/receptor interactions are available (see, e.g., Coligan et al. (2001) Current Protocols in Immunology, Vol. 4, John Wiley, Inc., N.Y.); methods for flow cytometry, including fluorescence-activated cell sorting (FACS), are available (see, e.g., Shapiro (2003) Practical Flow Cytometry, John Wiley and Sons, Hoboken, NJ); and fluorescent reagents suitable for modifying nucleic acids, including nucleic acid primers and probes, polypeptides, and antibodies, for use, for example, as diagnostic reagents, are available (Molecular Probes (2003) Catalogue, Molecular Probes, Inc., Eugene, Oreg.; Sigma-Aldrich (2003) Catalogue, St. Louis, Mo.).

Standard methods of histology of the immune system are described (see, e.g., Louis et al. (2002) Basic Histology: Text and Atlas, McGraw-Hill, New York, N.Y.).

Depletion of immune cells (CD4⁺ and CD8⁺ T-cells) may be effected by antibody-mediated elimination. For example, 250 μg of CD4- or CD8-specific antibodies may be injected weekly, and cell depletions verified using FACS and IHC analysis.

Software packages and databases for determining, e.g., antigenic fragments, leader sequences, protein folding, functional domains, glycosylation sites, and sequence alignments, are available (see, e.g., GCG Wisconsin Package (Accelrys, Inc., San Diego, Calif.); and DeCypherTM (TimeLogic Corp., Crystal Bay, Nev.).

Immunocompetent Balb/C or B-cell—deficient Balb/C mice may be obtained from The Jackson Lab., Bar Harbor, ME and may be used in accordance with standard procedures (see, e.g., Martin et al (2001) Infect. Immun., 69(11):7067-73 and Compton et al. (2004) Comp. Med. 54(6):681-89). Other mice strains suitable for the experimental work contemplated by the present disclosure are known to the skilled artisan and are generally available from The Jackson Lab. The skilled artisan is familiar with models and cell lines (e.g., models of inflammation) that may also be used in the practice of the present disclosure.

Serum IL-15 concentration levels and exposure levels may be determined by standard methods used in the art. For example, a serum exposure level assay can be performed by collecting whole blood (˜50 μL/mouse) from mouse tail snips into plain capillary tubes, separating serum and blood cells by centrifugation, and determining IL-15 exposure levels by standard ELISA kits (e.g., R&D Systems) and techniques. Alternatively, or in addition, the ELISA protocol described below (or a similar protocol) can be adapted to measure serum levels of human IL-15 as a means of determining in vivo half-life of a mutein or modified mutein.

IL-15 Protein: Human IL-15 was purchased from R&D Systems (Minneapolis, Minn., # 247-IL/CF, Accession #: P40933)

Human IL-15 Detection ELISA. A 96-well plate (Nunc Maxisorp #442404) may be coated overnight at 4° C. with 100 μL/well PBS +1 μg/mL anti-human IL-15 antibody (e.g., ATCC HB-12062, clone M111, Manassas, Vir.), washed 6×200 μL in DPBS-Tween 20 (Teknova #P0297), blocked in 200 μL/well PBS+5% BSA (Calbiochem #2960) for 2 hr at room temperature on a rocking platform, and washed as previously described. The samples may be serially diluted in PBS and 100 μL/well may be added to the assay plate. Samples may be run in duplicate or triplicate. As a positive control, purified human IL-15 may be spiked in, while buffer or conditioned media from a mock transfection may be used as a negative control, and both serially diluted. The samples may be incubated overnight at 4° C. on a rocking platform and then washed as previously described. 100 μL/well of PBS +anti-human-IL-15 antibody (e.g., ab7213; Abcam) may be added to each well, incubated for one hour at room temperature on a rocking platform, washed as previously described, and then 100 μL/well of donkey anti-rabbit IgG (H+L)-HRP (Jackson Immuno Research # 711-035-152, diluted 1:10,000) may be added and incubated for an additional 1 hr at room temperature on a rocking platform. The plate may be washed as described and developed with 100 μL/well of 1-Step Ultra TMB-ELISA (Pierce/Thermo #34029) for 1-5 mins, and then the reaction stopped with 100 μL/well Stop Solution (Life Technologies #SS04). The plate may be read on a Molecular Devices M2 plate reader at 450 nm.

Another ELISA format could include premade kits (e.g., following the manufacturer's recommended protocol in the Human IL-15 Quantikine ELISA Kit (R&D Systems #D1500, Minneapolis, MN)).

CTLL-2 Cell Proliferation Assay. Soman et al. (J Immunol Methods 348(1-2) 83-94 (2009 Aug. 31)) describe an optimized tetrazolium dye-based colorimetric cell proliferation assay of CTLL-2 cells using soluble CellTiter96 Aqueous One Reagent (Promega; Madison, Wis/) to quantitatively estimate IL-15 biological activity. CTLL-2 is an IL-2 dependent murine cell line.

A CTLL-2 cell proliferation assay substantively similar to that described by Soman et al. was used herein to determine IL-15 biological activity. Briefly, CTLL-2 cells (ATCC TIB-214, Manassas, Vir.) were cultured in RPMI 1640 (Life Technologies, 11875-093, Grand Island, N.Y.) supplemented with 10% FBS and 10% T-STIM (Corning #354115, Tewsbury, Mass.). The cells were maintained at 37° C. supplemented with 5% CO₂ at a density between 10,000 cells/mL and 100,000 cells/mL, and harvested when they were growing in a logarithmic phase (typically 2-3 weeks after thawing; cell viability≥95%) and washed four times with 20 mL of growth media without T-STIM (by centrifugation at 1000 rpm, 5 min). 25,000 cells/well in 100 μL of growth media without T-STIM were then aliquoted into clear 96-well tissue culture plates and returned to the incubator while the proteins were diluted. The IL-15 samples were diluted to an initial concentration of 8 ng/mL in the assay medium followed by serial two-fold dilutions, and then 100 μL added to the wells of a 96-well tissue culture plate and returned to the 37° C., 5% CO₂ incubator for 48 hr. After the 48 hr incubation period, CellTiter96® Aqueous One Solution was added (20 μL/well) and the suspension incubated for another 1-4 hr at 37° C. and 5% CO₂. The plate was read at 490 nm, and the background readings in the wells with medium were subtracted from the sample well read-outs.

M07e Cell Proliferation Assay. Kanakura et al. (Blood 76(4):706-15 (1990 Aug. 15)); Caliceti et al. (PLoS One 7(7): e41246. doi:10.1371/journal.pone.0041246 (2012)); and Zauner et al. (BioTechniques 20:905-13 (May 1996)) describe cell proliferation assays using M07e, a human leukemia megakaryocytic cell line whose proliferation is IL-3 or GM-CSF dependent. M07e cells may be purchased from DSMZ (DSMZ No. ACC 104; Braunschweig, Germany).

The M07e cell line may be cultured in RPMI 1640 medium (Gibco, Grand Island, N.Y.) supplemented with 10% FBS, rhGM-CSF (10 ng/mL) or rhIL-3 (10 ng/mL); alternatively, cells may be cultured in IMDM supplemented with 5% FCS and 10 ng/mL IL3. MTT [3-(4,5-dimethylthiazol-2-yl)-2,5-diphenyl tetrazolium bromide (Sigma) incorporation may be used to quantitate factor-induced proliferation of M07e cells. Briefly, triplicate aliquots of M07e cells may be cultured in flat-bottom microtiter plates (100 μL/well) for 72 hours at 37° C. MTT may be added for the final 4 hrs of culture (10 μL of a 5 mg/mL solution of MTT in PBS). At 72 hrs, 100 μL of acid isopropanol (0.04 N HCl in isopropanol) may be added to all wells, mixed, and the optical density measured on a micro ELISA plate reader at 540nm.

Purification of Wild Type and Mutein Human IL-15. An anti-human-IL-15 antibody (e.g. ATCC HB-12062, clone M111, Manassas, Va.) may be coupled to CNBr-activated Sepharose 4 Fast Flow (GE Healthcare #71-5000-15 AF, following the manufacturer's protocol) and equilibrated in PBS. 500 μL-1 mL of M111-sepaharose may be added per 100 mL of conditioned media contained in a glass Econo-Column (Bio-Rad, Hercules, Calif.) and incubated for 1-2 hours at room temperature on a rocking platform. The media may be run through the column via gravity flow, washed 1× with 1× PBS (pH 7.4), eluted with 0.1M glycine (pH 2.9) and neutralized with a 10% volume of 1M Tris buffer (pH 8.0). The protein may be concentrated and buffer exchanged into PBS (pH 7.4) using an Amicon Ultra Centrifugal Filter Device (Millipore, Billerica, Mass.; 5,000 kD molecular weight cutoff). Protein concentration may be determined by spectrophotometer at 280 nm.

SEC Analysis Proteins. Using a 1100 series HPLC (Agilent Technologies, Santa Clara, Calif.), 20-50 μg of protein may be injected onto a TSK3000sw column (Tosoh Biosciences, Tokyo, JP), equilibrated with PBS (pH 7.4), and run at a flow rate of 1 mL/min.

### Pegylation of IL-15

The PEG (NOF Corporation, Japan) may be diluted to a concentration of 10-100 mg/mL, in 50mM phosphate with 100mM NaCl at pH 4-8, and the human IL-15 may be diluted to a concentration of 2-10 mg/mL in PBS, pH 7.4. The final reaction mixture may include the PEG and human IL-15 at a 10:1 to 2:1-ratio range (PPA PEG:human IL-15), and sodium cyanoborohydride at a final concentration of 5-50mM. The reaction may be incubated from 4° C.-25° C. for 2-48 hrs. To select the desired protein species and/or buffer exchange, the pegylated protein may be fractionated via SEC (as previously described), or to eliminate most of the non-protein species in the pegylation reaction mixture and/or buffer exchange, the PEG-IL-15 reaction mixture may undergo an ultrafiltration step (e.g. a Millipore Labscale TFF system may be used with a regenerated cellulose (PLCGC) membrane, with a 5 kDa molecular weight cut off).

### Assays to Determine the Bioactivity of Modified Forms of IL-15

‘The present disclosure contemplates the use of any assays and methodologies known in the art for determining the bioactivity of the IL-15 molecules described herein. The assays described hereafter are representative, and not exclusionary.

CD8+/CD4+T-cell Assays. Activated primary human CD8+ and CD4+ T-cells secrete IFNγ, Granzyme B, Perforin and TNFα when treated with PEG-IL-15. The following protocol provides an exemplary assay for screening for the production of these cytokines. Human primary peripheral blood mononuclear cells (PBMCs) may be isolated according to any standard protocol (see, e.g., Fuss et al. (2009) Current Protocols in Immunology, Unit 7.1, John Wiley, Inc., N.Y.). 2.5 mL of PBMCs (at a cell density of 10 million cells/mL) may be cultured per well with complete RPMI, containing RPMI (Life Technologies; Carlsbad, Calif.), 10 mM HEPES (Life Technologies; Carlsbad, Calif.), 10% Fetal Calf Serum (Hyclone Thermo Fisher Scientific; Waltham, Mass.) and Penicillin/Streptomycin cocktail (Life Technologies; Carlsbad, Calif.), or in AIM-V serum-free media (Life Technologies #12055-083), in any standard tissue culture treated 6-well plate (BD; Franklin Lakes, N.J.) in a humidified 37° C. incubator with 5% CO₂. CD8+ and CD4+T-cells may be isolated using Miltenyi Biotec's MACS cell separation technology according to the manufacture's protocol (Miltenyi Biotech; Auburn, Calif.). The T-cells may be activated by coating a 24-well tissue culture plate (Costar #3526, Corning, N.Y.) with anti-CD3 and antiCD-28 antibodies (Affymetrix eBioscience; San Diego, Calif.) and by adding 3E6 cells/well in lml of AIM-V media. The cells may be grown for 3 days as described, then collected and resuspended in fresh AIM-V at a density of 2E6 cells/mL, and 250 μL/well aliquoted into a 96-well tissue culture plate (Falcon # 353072, Corning, N.Y.). Human PEG-IL-15 may be serially diluted and added to the wells at a final concentration of 1 μg/mL to 0.01 ng/ml; the cells may be incubated in a humidified 37° C. incubator with 5% CO₂ for 3 days. The media may then be collected and assayed for IFNγ, Granzyme B, Perforin and/or TNFα using a commercial ELISA kit and following the manufacture's protocol (e.g., Affymetrix Bioscience; San Diego, Calif. or R&D Systems, Minneapolis, Minn.)).

NK Cell Assays. Human NK cells may be isolated from the PBMC cells (protocol previously described; cultured in complete RPMI) and similarly isolated using Miltenyi Biotec's MACS cell separation technology according to the manufacture's protocol (Miltenyi Biotech; Auburn, Calif.). The cells may be grown and cultured (as described for the T-cells, using complete RPMI), plated in a 96-well tissue culture plate (Falcon #353072, Corning, N.Y.) at 5E5 cells/well in 250 μl of complete RPMI. After 1-3 days for growth, the media may be assayed as described for the T-cells.

### Tumor Models and Tumor Analysis

Any art-accepted tumor model, assay, and the like can be used to evaluate the effect of the IL-15 molecules described herein on various tumors. The tumor models and tumor analyses described hereafter are representative of those that can be utilized.

Syngeneic mouse tumor cells are injected subcutaneously or intradermally at 10⁴, 10⁵ or 10⁶ cells per tumor inoculation. Ep2 mammary carcinoma, CT26 colon carcinoma, PDV6 squamous carcinoma of the skin and 4T1 breast carcinoma models can be used (see, e.g., Langowski et al. (2006) Nature 442:461-465). Immunocompetent Balb/C or B-cell deficient Balb/C mice can be used. PEG-mIL-15 can be administered to the immunocompetent mice, while PEG-hIL-15 treatment can be in the B-cell deficient mice. Tumors are allowed to reach a size of 100-250 mm³ before treatment is started. IL-15, PEG-mIL-15, PEG-hIL-15, or buffer control is administered subcutaneously at a site distant from the tumor implantation. Tumor growth is typically monitored twice weekly using electronic calipers.

Tumor tissues and lymphatic organs are harvested at various endpoints to measure mRNA expression for a number of inflammatory markers and to perform immunohistochemistry for several inflammatory cell markers. The tissues are snap-frozen in liquid nitrogen and stored at −80° C. Primary tumor growth is typically monitored twice weekly using electronic calipers. Tumor volume may be calculated using the formula (width²×length/2) where length is the longer dimension. Tumors are allowed to reach a size of 90-250 mm³ before treatment is started.

### Example 1

Several series of pegylated rHuIL-15 molecules were prepared, and their activity was compared to that of unpegylated rHuIL-15. The present disclosure contemplates pegylated IL-15 molecules having one or more properties superior to those of unpegylated IL-15. Examples of such properties include potency comparable to or greater than unpegylated IL-15, extended half-life and/or other beneficial pharmacokinetic parameters (e.g., QW dosing sufficient to maintain serum exposure of ˜400/ng/mL), therapeutically acceptable stability, and efficient and cost-effective manufacturability.

Activated PEGs were obtained from NOF America Corp. (White Plains, N.Y.) and conjugated to rHuIL-15 using standard pegylation procedures and conditions (see, e.g., WO 2014/172392). As set forth in Table 1, several IL-15 PEG series comprising various PEG structures and sizes (MW) were generated and evaluated: Series 1: linear PEG; Series 2: 2-arm branched PEG; Series 3: 3-arm branched PEG; Series 4: bifunctional PEG; and Series 5: quad-functional (star) PEG. Unless otherwise indicated, in each series IL-15 was pegylated at its N-terminus.

Using the methods described above, EC50 values (ng/mL) were calculated to determine the potency of each molecule, and the percent of maximal activation of each molecule relative to unpegylated rHuIL-15 was determined (i.e., the maximal absorbance plateau measured at receptor saturation was calculated as a percentage of the unpegylated IL15 maximal absorbance plateau).

The data are set forth in Table 1

The data indicate that pegylated IL-15 molecules in Series 3, Series 5 and Series 2 (e.g., 20 kDa PEG) possess favorable potency. The increase in bioactivity of the Series 3 molecule relative to unpegylated IL-15 and Series 1 molecules was surprising, especially in view of the size of the PEG. For the particular Series 3 molecule in Table 1, referring to the formula below, x=y - 20 kDa, and w=10 kDa.
As described elsewhere herein, the present disclosure contemplates other PEG size distributions that would be considered Series 3 molecules (e.g., w=20 kDa and x=y=15 kDa).

In each of the Series 2 molecules set forth in Table 1, referring to the formula below, the total size of the PEG is attributable to the MW of x plus the MW of y, as the MW of the linker, examples of which are described herein, is negligible relative to that of x and y. By way of example, for the 20 kDa molecule in Table 1, x=y=10 kDa.

As indicated in Table 1, the potency of the 40 kDa, 60 kDa, and 80 kDa pegylated IL-15 molecules was dramatically less than that of the 20 kDa molecule.

As described elsewhere herein, the present disclosure contemplates other PEG size distributions that would be considered Series 2 molecules. By way of example, for a branched PEG IL-15 molecule comprising a 20 kDa PEG, x and y can each be 10 kDa in some embodiments, and x can be 5 kDa and y can be 15 kDa in other embodiments. Examples of linkers and PEGs are described herein.

For the particular Series 5 molecule in Table 1 (a quad-functional PEG IL-15 molecule), referring to the formula below, the A₁A₂A₃A₄ complex represents a PEG of 20 kDa that is covalently attached to each of the four IL-15. Each A₁, A₂, A₃ and A₄ is 5 kDa. The PEG may optionally be attached to one or more of the IL-15 through a linker.

The quad-functional PEG Series 5 molecule possessed reasonable potency, but such star PEGs present challenges associated with manufacturability and stability (data not shown).

Particular embodiments of this invention are described herein, including the best mode known to the inventors for carrying out the invention. Upon reading the foregoing, description, variations of the disclosed embodiments may become apparent to individuals working in the art, and it is expected that those skilled artisans may employ such variations as appropriate. Accordingly, it is intended that the invention be practiced otherwise than as specifically described herein, and that the invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

All publications, patent applications, accession numbers, and other references cited in this specification are herein incorporated by reference as if each individual publication or patent application was specifically and individually indicated to be incorporated by reference.

What is claimed:

1. A multi-arm PEG IL-15 molecule having the formula: 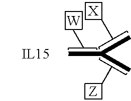
wherein x, w and z represent components of a PEG, and the IL-15 is covalently attached, optionally via a linker, to w.

2. The multi-arm PEG IL-15 molecule of claim 1, wherein the MW of each of x, w and z is the same.

3. The multi-arm PEG IL-15 molecule of claim 1, wherein the MW of at least one of x, w and z is different.

4. The multi-arm PEG IL-15 molecule of claim 3, wherein the MW of each of x and z is the same.

5. The multi-arm PEG IL-15 molecule of claim 3, wherein the MW of each of x and z is the different.

6. The multi-arm PEG IL-15 molecule of claim 1, wherein the MW of the PEG is from 7.5 kDa to 80 kDa.

7. The multi-arm PEG IL-15 molecule of claim 1, wherein the MW of the PEG is from 30 kDa to 60 kDa.

8. The multi-arm PEG IL-15 molecule of claim 1, wherein the MW of the PEG is about 50 kDa.

9. The multi-arm PEG IL-15 molecule of claim 8, wherein the MW of each of x and z is 20 kDa, and the MW of w is 10 kDa.

10. The multi-arm PEG IL-15 molecule of claim 1, wherein the IL-15 is covalently attached via a linker to w.

11. A branched PEG IL-15 molecule having the formula: 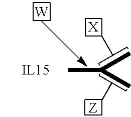
wherein x and z represent components of a PEG, and the IL-15 is covalently attached to the PEG via a linker w.

12. The branched PEG IL-15 molecule of claim 11, wherein the MW of the PEG is from 5 kDa to 80 kDa.

13. The branched PEG IL-15 molecule of claim 11, wherein the MW of the PEG is about 20 kDa.

14. The branched PEG IL-15 molecule of claim 13, wherein the MW of each of x and z is 10 kDa.

15. The branched PEG IL-15 molecule of claim 11, wherein the MW of the PEG is about 40 kDa.

16. The branched PEG IL-15 molecule of claim 15, wherein the MW of each of x and z is 20 kDa.

17. The branched PEG IL-15 molecule of clai 11, wherein the MW of the PEG is about 60 kDa.

18. The branched PEG IL-15 molecule of claim 17, wherein the MW of each of x and z is 30 kDa.

19. The branched PEG IL-15 molecule of claim 11, wherein the MW of the PEG is about 80 kDa.

20. The branched PEG IL-15 molecule of claim 19, wherein the MW of each of x and z is 40 kDa.

21. The PEG IL-15 molecule of any one of claims 1-20, wherein the IL-15 is human IL-15.

22. The PEG IL-15 molecule of any one of claims 1-20, wherein the IL-15 is an IL-15 mutein.

23. The PEG IL-15 molecule of claim 22, comprising:
a) a Helix A, b) an A/B Inter-helix Junction, c) a Helix B, d) a B/C Inter-helix Junction, e) a Helix C, f) a C/D Inter-helix Junction and g) a Helix D; and wherein the peptide further comprises at least one amino acid substitution comprising:
substitution of at least one amino acid residue of Helix A other than amino acid residues 2 (W), 4-12 (NVISDLKKI; SEQ ID NO:7), or 16 (I); or
substitution of at least one amino acid residue of the A/B Inter-helix Junction other than amino acid residues 30 (D) or 31 (V); or
substitution of at least one amino acid residue of Helix B other than amino acid residues 32 (H), 35 (C), 40 (M), 42-44 (CFL), 47 (L) or 50 (I); or
substitution of at least one amino acid residue of the B/C Inter-helix Junction; or
substitution of at least one amino acid residue of Helix C other than amino acid residues 59 (I), 61-66 (DTVENL; SEQ ID NO:8), or 68-70 (ILA); or
substitution of at least one amino acid residue of the C/D Inter-helix Junction other than amino acid residues 85 (C) or 88 (C); or
substitution of at least one amino acid residue of Helix D other than amino acid residues 99 (F), 100 (L), 103 (F), or 105-112 (HIVQMFIN; SEQ ID NO:9).

24. The PEG IL-15 molecule of claim 23, wherein the at least one amino acid substitution is a conservative substitution.

25. The PEG IL-15 molecule of claim 23, wherein the at least one amino acid substitution is at one of the following positions: 1, 3, 13-15, 17-29, 33, 34, 36-39, 41, 45, 48, 49, 51-58, 60, 67, 71-84, 86, 87, 89-98, 101, 102, 104, 113, or 114.

26. The PEG IL-15 molecule of claim 25, wherein the at least one amino acid substitution comprises substitution of a tyrosine for at least one of the amino acid residues at the following positions: 1, 3, 13-15, 17-25, 27-29, 33, 34, 36-39, 41, 45, 48, 49, 51-58, 60, 67, 71-84, 86, 87, 89-98, 101, 102, 104, 113, or 114.

27. The PEG IL-15 molecule of claim 25, wherein the at least one amino acid substitution comprises substitution of a cysteine for at least one of the amino acid residues at the following positions: 1, 3, 13-15, 17-25, 27-29, 33, 34, 36-39, 45, 48, 49, 51-56, 58, 60, 67, 72-84, 86, 87, 89-98, 101, 102, 104, 113, or 114.

28. The PEG IL-15 molecule of claim 25, wherein the at least one amino acid substitution comprises substitution of an N-X-S glycosylation motif for at least one of the amino acid residues at the following positions: 1, 13-15, 17-22, 27-29, 34, 36, 48, 49, 51-58, 60, 72-82, 84, 87, 89-98, 102, or 104,
wherein the asparagine of the N-X-S glycosylation motif represents the amino acid position.

29. The PEG IL-15 molecule of claim 25, wherein the at least one amino acid substitution comprises substitution of an N-X-T glycosylation motif for at least one of the amino acid residues at the following positions: 1, 13-15, 17-22, 29, 34, 36, 48, 49, 51-58, 60, 71-78, 80-82, 84, 87, 89-98, or 102,
wherein the asparagine of the N-X-T glycosylation motif represents the amino acid position.

30. The PEG IL-15 molecule of any one of claims 22-29, wherein the IL-15 is produced recombinantly.

31. A pharmaceutical composition, comprising a peptide of claim 1, 11, 22 or 23, and a pharmaceutically acceptable diluent, carrier or excipient.

32. The pharmaceutical composition of claim 31, wherein the excipient is an isotonic injection solution.

33. The pharmaceutical composition of claim 31, wherein the pharmaceutical composition is suitable for human administration.

34. The pharmaceutical composition of claim 31, further comprising at least one additional prophylactic or therapeutic agent.

35. A sterile container comprising the pharmaceutical composition of claim 31.

36. The sterile container of claim 35, wherein the sterile container is a syringe.

37. A kit comprising the sterile container of claim 36.

38. The kit of claim 37, further comprising a second sterile container comprising at least one additional prophylactic or therapeutic agent.

39. A method of treating or preventing a disease, disorder or condition in a subject, comprising administering to the subject a therapeutically effective amount of a peptide of claim 1, 11, 22 or 23.

40. The method of claim 39, wherein the disease, disorder or condition is a proliferative disorder.

41. The method of claim 40, wherein the proliferative disorder is a cancer.

42. The method of claim 41, wherein the cancer is a solid tumor or a hematological disorder.

43. The method of claim 39, wherein the disease, disorder or condition is an immune or inflammatory disorder.

44. The method of claim 43, wherein the immune or inflammatory disorder is selected from the group consisting of inflammatory bowel disease, psoriasis, rheumatoid arthritis, multiple sclerosis, and Alzheimer's disease.

45. The method of claim 39, wherein the disease, disorder or condition is a viral disorder.

46. The method of claim 45, wherein the viral disorder is selected from the group consisting of human immunodeficiency virus, hepatitis B virus, hepatitis C virus and cytomegalovirus.

47. The method of claim 39, wherein the subject is human.

48. The method of claim 39, wherein the administering is by parenteral injection.

49. The method of claim 48, wherein the parenteral injection is subcutaneous.

50. The method of claim 39, further comprising administering at least one additional prophylactic or therapeutic agent.

51. A process for preparing the PEG IL-15 molecule of claim 1, 11, 22 or 23, comprising the step of:
reacting IL-15 with an activated PEG linker under conditions in which the linker covalently attaches to one amino acid residue of the IL-15.

52. The process of claim 51, wherein the activated PEG linker is selected from the group consisting of succinimidylcarbonate-PEG, PEG-butyraldehyde, PEG-pentaldehyde, PEG-amido-propionaldehyde, PEG-urethano-propioaldehyde, and PEG-propylaldehyde.

53. A pegylated interleukin-15 molecule, comprising the formula:
(IL-15-L)ₐ-PEG,
wherein a is 2-4 and each L, if present, is a linker covalently attaching the PEG molecule to i) an amino group of a single amino acid residue of each IL-15, wherein the amino group of the single amino acid residue is the alpha amino group of the N-terminal amino acid residue or the epsilon amino group of a lysine amino acid residue, or ii) an N-glycosylation site.

54. The pegylated interleukin-15 molecule of claim 53, wherein a=2.

55. The pegylated interleukin-15 molecule of claim 53, wherein a=3.

56. The pegylated interleukin-15 molecule of claim 53, wherein a=4.

57. The pegylated interleukin-15 molecule of claim 53, wherein the amino group of the single amino acid residue is the alpha amino group of the N-terminal amino acid residue.

58. The pegylated interleukin-15 molecule of claim 53, wherein the amino group of the single amino acid residue is the epsilon amino group of a lysine amino acid residue.

59. The pegylated interleukin-15 molecule of claim 53, wherein the N-glycosylation site comprises an N-X-S motif.

60. The pegylated interleukin-15 molecule of claim 53, wherein the N-glycosylation site comprises an N-X-T motif.

61. The pegylated interleukin-15 molecule of any one of claims 53-60, wherein the PEG has a molecular weight of from 5 kDa to 40 kDa.

62. The pegylated interleukin-15 molecule of claim 61, wherein the PEG has a molecular weight of about 10 kDa.

63. The pegylated interleukin-15 molecule of claim 61, wherein the PEG has a molecular weight of about 20 kDa.

64. The pegylated interleukin-15 molecule of claim 61, wherein the PEG has a molecular weight of about 30 kDa.

65. A pegylated IL-15 molecule (PEG-IL-15), comprising at least one branched or multi-arm polyethylene glycol (PEG) molecule covalently attached to a single amino acid residue of IL-15, wherein the amino acid residue is i) the alpha amino group of the N-terminal amino acid residue, ii) the epsilon amino group of a lysine amino acid residue, or iii) an N-glycosylation site; and wherein the PEG is optionally covalently attached to the IL-15 through a linker.

66. The PEG-IL-15 of claim 65, comprising the formula: (PEG)_{b}-L-NH-IL-15, wherein the PEG is a branched polyethylene glycol of molecular weight between 5 kDa and 80 kDa; b is 1-9; and L is an optionally present linker moiety attaching the PEG to the single amino acid residue.

67. The PEG-IL-15 of claim 65, comprising the formula: (PEG)_{b}-L-NH-IL-15, wherein the PEG is a multi-arm polyethylene glycol of molecular weight between 50 kDa and 80 kDa; b is 1-9; and L is an optionally present linker moiety attaching the PEG to the single amino acid residue.

68. The PEG-IL-15 of any one of claims 65-67, wherein the PEG is attached to the alpha amino group of the N-terminal amino acid residue.

69. The PEG-IL-15 of any one of claims 65-67, wherein the PEG is attached to the epsilon amino group of a lysine amino acid residue.

70. The PEG-IL-15 of any one of claims 65-67, wherein the PEG is attached to an N-glycosylation site.

71. The PEG-IL-15 of claim 70, wherein the N-glycosylation site comprises an N-X-S motif.

72. The PEG-IL-15 of claim 70, wherein the N-glycosylation site comprises an N-X-T motif.

73. The PEG-IL-15 of claim 66 or 67, wherein the linker moiety is covalently attached to the single amino acid residue.

74. The PEG-IL-15 of claim 66 or 67, wherein b is 1 and L is a C₂-C₁₂ alkyl.

75. The PEG-IL-15 of claim 66 or 67, wherein the linker is an activated PEG linker selected from the group consisting of succinimidylcarbonate-PEG, PEG-butyraldehyde, PEG-pentaldehyde, PEG-amido-propionaldehyde, PEG-urethano-propioaldehyde, and PEG-propylaldehyde.

76. The PEG-IL-15 of any one of claims 65-75, wherein the PEG has a molecular weight of from 5 kDa to 80 kDa.
